(19) United States
(12) Patent Application Publication
(10) Pub. No.: US 2017/0246163 A1
(43) Pub. Date: Aug. 31, 2017

(54) METHODS OF IMPROVING BALANCE, GAIT OR BOTH IN PATIENTS WITH NEUROLOGICAL DISEASE

(71) Applicant: Axovant Sciences GmbH, Basel (CH)

(72) Inventors: Shankar RAMASWAMY, Cincinnati, OH (US); Lawrence Tim FRIEDHOFF, Rivervale, NJ (US); Ilise LOMBARDO, New York, NY (US); Ebenezer ASARE, New Haven, CT (US)

(21) Appl. No.: 15/443,839

(22) Filed: Feb. 27, 2017

Related U.S. Application Data
(60) Provisional application No. 62/299,725, filed on Feb. 25, 2016.
(60) Provisional application No. 62/406,821, filed on Oct. 11, 2016.

(51) Int. Cl. A61K 31/496

(52) U.S. Cl. CPC A61K 31/496

(57) ABSTRACT

Disclosed herein are compositions and methods for reducing the frequency of falls, improving balance, improving gait, or some combination thereof in subjects with neurodegenerative disease.

### CROSS REFERENCE TO RELATED APPLICATIONS

This application claims the priority benefit under 35 U.S.C. 119(e) of U.S. Provisional Application No. 62/299,725 filed Feb. 25, 2016 and U.S. Provisional Application No. 62/406,821 filed Oct. 11, 2016, the disclosures of which are incorporated by reference in their entireties.

### FIELD

The disclosure generally refers to compositions and methods to improve balance, gait or both in patients with neurological disease.

### SUMMARY

Embodiments herein provide methods of improving balance, gait, or both in a subject in need thereof, comprising administering to said subject a therapeutically effective amount of intepirdine or pharmaceutically acceptable salt, hydrate or solvate thereof.

Embodiments herein provide methods of decreasing falls, improving balance, improving gait, or some combination hereof in a subject in need thereof, comprising administering to said subject a therapeutically effective amount of intepirdine or pharmaceutically acceptable salt, hydrate or solvate thereof.

Embodiments herein provide methods of improving the balance, gait, or both in a subject in need thereof, wherein the subject has a neurodegenerative disease and has not experienced an increase in falls, comprising administering to said subject a therapeutically effective amount of intepirdine, or pharmaceutically acceptable salts, hydrates or solvates thereof.

In some embodiments, the subject has experienced less than one fall per week prior to administering the therapeutically effective amount of intepirdine, or pharmaceutically acceptable salts, hydrates or solvates thereof. In some embodiments, the subject has experienced less than one fall per month prior to administering the therapeutically effective amount of intepirdine, or pharmaceutically acceptable salts, hydrates or solvates thereof.

In some embodiments, the subject exhibits Postural Instability and Gait Difficulty (PIGD) subtype assessed by the MDS-UPDRS prior to administering to the subject a therapeutically effective amount of intepirdine or pharmaceutically acceptable salts, hydrates or solvates thereof.

In some embodiments, the neurodegenerative disease is selected from the group consisting of Alzheimer's disease, Creutzfeldt-Jakob disease, Dementia with Lewy bodies, frontotemporal dementia, Huntington's disease, levodopa-induced dyskinesia, Lewy body dementia, mixed Alzheimer's disease and cardiovascular disease, multiple sclerosis, orthostatic hypotension in Parkinson's disease, Parkinson's disease, Parkinson's disease dementia, progressive supranuclear palsy, vascular dementia, and any combination thereof.

In some embodiments, the subject is aged between about 50 and about 95 years of age. In some embodiments, the subject is receiving stable therapy for a movement disorder, a balance disorder, a cognitive disorder, or any combination thereof. In some embodiments, the subject is receiving a daily dose of about 1 mg to about 25 mg of donepezil.

In some embodiments, the therapeutically effective amount of intepirdine is between about 0.001 mg and 70 mg. In some embodiments, the therapeutically effective amount of intepirdine is about 15 mg. In some embodiments, the therapeutically effective amount of intepirdine is about 35 mg. In some embodiments, the therapeutically effective amount of intepirdine is about 70 mg.

In some embodiments, the administration of a therapeutically effective amount of intepirdine, or pharmaceutically acceptable salts, hydrates or solvates thereof, results in an improvement in quantitative measures of balance and gait. In some embodiments, balance and gait are changed from baseline in the individual item scores calculated by Opal APDM sensors during performance of the mini-BESTest battery.

In some embodiments, the administration of a therapeutically effective amount of intepirdine, or pharmaceutically acceptable salts, hydrates or solvates thereof, results in an improvement in balance. In some embodiments, balance is assessed by a change from baseline in the root mean square (RMS) of mediolateral sway, measured using Opal APDM sensors during the mini-BESTest evaluation.

In some embodiments, the administration of a therapeutically effective amount of intepirdine, or pharmaceutically acceptable salts, hydrates or solvates thereof, results in an improvement in movement and balance. In some embodiments, movement and balance is measured by the subjects' Motor Disorder Society Unified Parkinson's Disease Rating Scale (MDS-UPDRS), MDS-UPDRS Part III sub-score, MDS-UPDRS PIGD score, or any combination thereof.

In some embodiments, the administration of a therapeutically effective amount of intepirdine, or pharmaceutically acceptable salts, hydrates or solvates thereof, results in an improvement in balance confidence. In some embodiments, balance confidence is assessed by a change from baseline in the Activities-Specific Balance Confidence (ABC) scale.

In some embodiments, the administration of a therapeutically effective amount of intepirdine, or pharmaceutically acceptable salts, hydrates or solvates thereof, results in an improvement in gait. In some embodiments, gait is assessed by a change from baseline in step time variability, stride time variability, stride length variability, step length variability, gait speed or any combination thereof, under normal and dual-task walking conditions measured on a GAITRite electronic walkway system.

In some embodiments, the administration of a therapeutically effective amount of intepirdine, or pharmaceutically acceptable salts, hydrates or solvates thereof, results in an improvement in gait speed. In some embodiments, gait speed is assessed by quantitatively measuring the functional mobility, evaluated under normal and dual-task walking conditions on an electronic walkway system.

In some embodiments, the wherein administration of a therapeutically effective amount of intepirdine, or pharmaceutically acceptable salts, hydrates or solvates thereof, results in an improvement in gait variability. In some embodiments, gait variability is changed from baseline under normal and dual-task walking conditions measured on an electronic walkway system.

In some embodiments, the administration of a therapeutically effective amount of intepirdine, or pharmaceutically acceptable salts, hydrates or solvates thereof, results in a decrease in freezing of gait. In some embodiments, freezing of gait is measured by rapid 360-degree turns test, rapid full turns test, walking under normal and dual task trajectory, walking under normal and dual task conditions, change in baseline in Freezing ratio, measured using APDM Opal sensors, GAITRite analysis, and a combination thereof.

In some embodiments, the administration of a therapeutically effective amount of intepirdine, or pharmaceutically acceptable salts, hydrates or solvates thereof, results in an improvement in dynamic gait and balance. In some embodiments, dynamic gait and balance is measured by a change from baseline in measures of dynamic gait and balance assessed by data reduction and factor analysis using APDM Opal sensors during a mini-BESTest.

In some embodiments, the administration of a therapeutically effective amount of intepirdine, or pharmaceutically acceptable salts, hydrates or solvates thereof, results in an improvement in functional mobility. In some embodiments, the administration of a therapeutically effective amount of intepirdine, or pharmaceutically acceptable salts, hydrates or solvates thereof, results in an improvement in the subjects' Motor Disorder Society Unified Parkinson's Disease Rating Scale (MDS-UPDRS), MDS-UPDRS Part III sub-score, MDS-UPDRS PIGD score, or any combination thereof.

### BRIEF DESCRIPTION OF THE FIGURES

FIG. 1 depicts the design of a Phase 2, double-blind, randomized, placebo-controlled parallel group study evaluating the effects of intepirdine on balance and gait in patients with a neurodegenerative disease.

### DETAILED DESCRIPTION

Before the present methods are described, it is to be understood that this disclosure is not limited to the particular processes, compositions, or methodologies described, as these may vary. Moreover, the processes, compositions, and methodologies described in particular embodiments are interchangeable. Therefore, for example, a composition, dosages regimen, route of administration, and so on described in a particular embodiments may be used in any of the methods described in other particular embodiments. It is also to be understood that the terminology used in the description is for the purpose of describing the particular versions or embodiments only, and is not intended to the limit the scope of the present disclosure which will be limited only by the appended claims. Unless defined otherwise, all technical and scientific terms used herein have the same meanings as commonly understood by one of the ordinary skill in the art. All publications and references mentioned herein are incorporated by reference.

As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic agents, pharmaceutical agents or equivalents thereof known to those skilled in the art, and so forth. Thus, for example, a reference to “a host cell” includes a plurality of such host cells, and a reference to “an antibody” is a reference to one or more antibodies and equivalents thereof known to those skilled in the art, and so forth.

As used herein, the term “about” means plus or minus 10% of the numerical value of the number with which it is being used. Therefore, about 50% means in the range of 45%-55%.

As used herein, the term “and/or” is included for convenience and means both “and” and “or”. A phrase using the term “and/or” is considered two separate embodiments, one embodiment containing “and” and the other embodiment containing “or”. For example, the phrase “improving balance and/or gait” should be considered as the separate embodiments of “improving balance and gait” and “improving balance or gait”. In some embodiments, the phrase “improving balance and/or gait” can be considered as the embodiment of “improving balance, gait, or both.”

“Administering” when used in conjunction with a therapeutic means to administer a therapeutic directly or indirectly into or onto a target tissue to administer a therapeutic to a patient whereby the therapeutic positively impacts the tissue to which it is targeted. “Administering” a composition may be accomplished by oral administration, injection, infusion, inhalation, absorption or by any method in combination with other known techniques. “Administering” may include the act of self-administration or administration by another person such as a health care provider.

As used herein, the term “daily dose” refers to the amount of intepirdine or pharmaceutically acceptable salts, hydrates or solvates thereof, per day that is administered or prescribed to a patient. This amount can be administered in multiple unit doses or in a single unit dose, at a single time during the day or at multiple times during the day.

The term “improves” is used to convey that the present disclosure changes either the appearance, form, characteristics, structure, function, or physical attributes of the tissue to which it is being provided, applied or administered. “Improves” may also refer to the overall physical state of an individual to whom an active agent has been administered. For example, the overall physical state of an individual may “improve” if one or more symptoms of the disease, condition or disorder are alleviated by administration of an active agent.

In some embodiments, the compositions and methods may be utilized with or on a subject in need of such treatment, which may also be referred to as “in need thereof.” As used herein, the phrase “in need thereof” means that the subject has been identified as having a need for the particular method or treatment and that the treatment has been given to the subject for that particular purpose.

As used herein, the term “patient” and “subject” are interchangeable and may be taken to mean any living organism, which may be treated with compounds of the present disclosure. As such, the terms “patient” and “subject” may include, but is not limited to, any non-human mammal, primate or human. In some embodiments, the “patient” or “subject” is an adult, child, infant, or fetus. In some embodiments, the “patient” or “subject” is a human. In some embodiments, the “patient” or “subject” is a mammal, such as mice, rats, other rodents, rabbits, dogs, cats, swine, cattle, sheep, horses, primates, or humans.

As used herein, the phrase “pharmaceutically acceptable” refers to those compounds, materials, compositions, dosage forms, or combinations thereof, which are, within the scope of sound medical judgment, suitable for use in contact with the tissues of human beings and animals without excessive toxicity, irritation, allergic response, or other problem or complication, commensurate with a reasonable benefit/risk ratio.

By “pharmaceutical formulation” it is further meant that the carrier, solvent, excipients and salt must be compatible with the active ingredient of the formulation (e.g. a compound described herein). It is understood by those of ordinary skill in this art that the terms “pharmaceutical formulation” and “pharmaceutical composition” are generally interchangeable, and they are so used for the purposes of this application.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic. The present disclosure includes pharmaceutically acceptable salts of any compound(s) described herein.

Pharmaceutically acceptable salts can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile, and the like. Lists of suitable salts are found in Remington's Pharmaceutical Sciences, 18th ed., Mack Publishing Company, Easton, Pa., USA, p. 1445 (1990).

Since prodrugs are known to enhance numerous desirable qualities of pharmaceuticals (e.g., solubility, bioavailability, manufacturing, etc.) the compounds described herein can be delivered in prodrug form and can be administered in this form for the treatment of disease. “Prodrugs” are intended to include any covalently bonded carriers that release an active parent drug of described herein in vivo when such prodrug is administered to a mammalian subject. Prodrugs are prepared by modifying functional groups present in the compound in such a way that the modifications are cleaved, either in routine manipulation or in vivo, to the parent compound. Prodrugs include compounds described herein wherein a hydroxy, amino, or sulfhydryl group is bonded to any group that, when the prodrug is administered to a mammalian subject, it cleaves to form a free hydroxyl, free amino, or free sulfhydryl group, respectively. Examples of prodrugs include, but are not limited to, acetate, formate, and benzoate derivatives of alcohol and amine functional groups in the compounds described herein.

The terms “therapeutically effective amount” or “therapeutic dose” is used herein are interchangeable and may refer to the amount of an active agent or pharmaceutical compound or composition that elicits a clinical, biological or medicinal response in a tissue, system, animal, individual or human that is being sought by a researcher, veterinarian, medical doctor or other clinical professional. A clinical, biological or medical response may include, for example, one or more of the following: (1) preventing a disease, condition or disorder in an individual that may be predisposed to the disease, condition or disorder but does not yet experience or display pathology or symptoms of the disease, condition or disorder, (2) inhibiting a disease, condition or disorder in an individual that is experiencing or displaying the pathology or symptoms of the disease, condition or disorder or arresting further development of the pathology, symptoms, or both of the disease, condition or disorder, and (3) ameliorating a disease, condition or disorder in an individual that is experiencing or exhibiting the pathology or symptoms of the disease, condition or disorder or reversing the pathology, symptoms, or both experienced or exhibited by the individual.

As used herein, “treating” or “treatment” includes any effect e.g., lessening, reducing, modulating, or eliminating, that results in the improvement of the condition, disease, disorder, etc. “Treating” or “treatment” of a disease state means the treatment of a disease-state in a mammal, particularly in a human, and include: (a) inhibiting an existing disease-state, i.e., arresting its development or its clinical symptoms; (c) relieving the disease-state, i.e., causing regression of the disease state; or some combination thereof.

Disclosed herein are methods of improving balance, gait, or both in a subject in need thereof, comprising administering to the subject a therapeutically effective amount of a 5-HT₆ receptor antagonist. In some embodiments, the methods comprise decreasing falls, improving balance, improving gait, or a combination thereof in a subject in need thereof. In some embodiments, the subject has not had an increase in falls prior to the administering of the 5-HT₆ receptor antagonist. In some embodiments, the subject has a neurodegenerative disease. In some embodiments, the methods comprise administering to the subject a combination of a 5-HT₆ receptor antagonist and a second therapeutic agent such as, but not limited to, a cholinesterase inhibitor.

As used herein, the term “balance” is defined as the ability of a subject to maintain the position of their center of mass within their support base, e.g. a subject maintaining their balance while standing upright will keep their center of mass between the positions of their feet. As used herein, the term “gait” is defined at the pattern of movement of the limbs of a subject while engaged in locomotion, i.e. walking or running. In some embodiments, the ability of a subject to properly maintain and control balance and gait are important for a range of basic functions, including, but not limited to, standing, sitting, and walking. In some embodiments, subjects unable to properly maintain or control balance, gait, or both are at an increased risk of one or more falls. As used herein, the term “fall” is defined as an event which results in a person coming to rest inadvertently on the ground or floor or other lower level. In some embodiments, one or more neurological diseases in a subject may degrade or limit the subject's ability to maintain proper balance, gait, or both. In some embodiments, one or more neurological diseases in a subject may increase the subject risk of falling.

In some embodiments, the 5-HT₆ receptor antagonist is 3-phenylsulfonyl-8-piperazinyl-1yl-quinoline and its hydrochloride salt. As used herein, 3-phenylsulfonyl-8-piperazinyl-1yl-quinoline may also be referred to as RVT-101, SB742457, or intepirdine. Intepirdine is a potent and selective 5HT₆ receptor antagonist. As used herein intepirdine may be 3-phenylsulfonyl-8-piperazinyl-1yl-quinoline or pharmaceutically acceptable salts, hydrates, solvates, or polymorphs, thereof. In some embodiments, intepirdine may be a compound of the foimula (I):

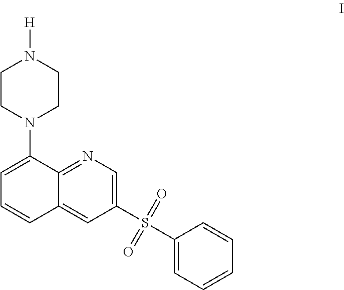 [SMILES: O=S(=O)(c1ccccc1)c1cnc2c(N3CCNCC3)cccc2c1]

or pharmaceutically acceptable salts, hydrates, solvates, or polymorphs, thereof.

In some embodiments, the method may comprise administering a therapeutically effective amount of intepirdine or pharmaceutically acceptable salts, hydrates or solvates thereof. In some embodiments, the method may consist essentially of administering a therapeutically effective amount of intepirdine, or pharmaceutically acceptable salts, hydrates or solvates thereof. In some embodiments, the method may consist of administering a therapeutically effective amount of intepirdine or pharmaceutically acceptable salts, hydrates or solvates thereof. The term “comprising” means “including, but not limited to.” The term “consisting essentially of” means the method or composition includes the steps or components specifically recited, and may also include those that do not materially affect the basic and novel characteristics of the present disclosure. The term “consisting of” means the method or composition includes only the steps or components specifically recited.

In some embodiments, the method comprises administering intepirdine, or pharmaceutically acceptable salts, hydrates or solvates thereof, as a pharmaceutical composition. In some embodiments, the pharmaceutical composition comprises intepirdine, or pharmaceutically acceptable salts, hydrates or solvates thereof, and a pharmaceutically acceptable excipient. In some embodiments, the pharmaceutical composition comprises a therapeutically effective amount of intepirdine, or pharmaceutically acceptable salts, hydrates or solvates thereof. In some embodiments, the pharmaceutical composition comprises a daily dose of intepirdine, or pharmaceutically acceptable salts, hydrates or solvates thereof.

In some embodiments, the subject is receiving stable therapy for a movement disorder, a balance disorder, a cognitive disorder, or any combination thereof. In some embodiments, the subject is on stable cholinesterase inhibitor therapy. In some embodiments, the subject has been on a stable background cholinesterase inhibitor therapy for at least 60 days prior to treatment and may have received a cholinesterase inhibitor at any dose for at least four months prior to prior to administering intepirdine. In some embodiments the acetylcholinesterase inhibitor is donepezil, or pharmaceutically acceptable salts, hydrates or solvates thereof. In some embodiments, donepezil, or pharmaceutically acceptable salts, hydrates or solvates thereof is administered to a subject in need thereof, in a therapeutically effective amount.

In some embodiments, the subject has a neurodegenerative disease. In some embodiments, neurodegenerative diseases include, but are not limited to, Alzheimer's disease, Creutzfeldt-Jakob disease, Dementia with Lewy bodies, frontotemporal dementia, Huntington's disease, levodopa-induced dyskinesia, Lewy body dementia, mixed Alzheimer's disease and cardiovascular disease, multiple sclerosis, orthostatic hypotension in Parkinson's disease, Parkinson's disease, Parkinson's disease dementia, progressive supranuclear palsy, vascular dementia, and any combination thereof. As used herein, “dementia” is a broad category of neurodegenerative diseases that cause a decrease in a range of mental and physical capacities over time, including, but not limited to cognition, memory, language, motivation, movement, and behavior. In some embodiments, the neurodegenerative disease is classified as mild, moderate, severe, mild to moderate, or moderate to severe. In some embodiments, the dementia is classified as mild, moderate, severe, mild to moderate, or moderate to severe. In some embodiments, the dementia is of the Alzheimer's type. In some embodiments, the dementia is associated with Parkinson's disease. In some embodiments, the methods disclosed herein can be used in the treatment of a neurodegenerative disease. In some embodiments, the methods disclosed herein can be used in the treatment of dementia. In some embodiments, the methods disclosed herein can be used to treat mild, moderate, severe, mild to moderate, or moderate to severe dementia. In some embodiments, the methods disclosed herein can be used to treat dementia of the Alzheimer's type. In some embodiments, the methods disclosed herein can be used to treat dementia associated with Parkinson's disease.

In some embodiments, the subject has cognitive impairment. In some embodiments, the subject is elderly. In some embodiments, the subject has Parkinson's disease. In some embodiments the subject is diagnosed with Parkinson's disease. The dementia of Parkinson's disease is purportedly characterized by impairments in executive function, memory retrieval, and attention, in patients with an established diagnosis of Parkinson's disease. The diagnosis of the dementia of Parkinson's disease can be made reliably in patients in whom a progressive dementia syndrome occurs (without the necessity to document the specific deficits described above) at least 2 years after a diagnosis of Parkinson's disease has been made, and in whom other causes of dementia have been ruled out. Falls in subjects with Parkinson's disease are associated with significant injury, disability, hospitalization, and reduced quality of life. The causes of falls in PD are multifactorial and extend beyond progressive motor impairment and dyskinesia which are characteristics of Parkinson's disease. In some embodiments, the subject has Parkinson's disease dementia. In some embodiments the subject is diagnosed with Parkinson's disease dementia.

Parkinson's disease (PD) is a progressive neurodegenerative disease characterized by the cardinal clinical features of tremor, bradykinesia, rigidity, and postural instability. PD is estimated to affect 1% of people over 60 years of age, which represents more than 1 million individuals in North America alone. The defining pathological feature of PD is the accumulation of Lewy bodies, which are toxic neuronal inclusions composed primarily of alpha synuclein, ubiquitin and other proteins, in various regions of the brain. Traditionally considered a disorder of movement secondary to nigrostriatal dopaminergic cell loss, PD has now been recognized as being associated with significant non-motor features including depression, psychosis, anxiety, and sleep disturbances. Cognitive impairment is another significant impairment seen in PD. A longitudinal study revealed that 83% of patients who survived for 20 years with PD developed dementia. Patients with PD-related dementia account for 3-4% of all cases of dementia. Parkinson's disease dementia (PDD), also termed major neurocognitive disorder (NCD) due to Parkinson's Disease, is defined by the Diagnostic and Statistical Manual of Mental Disorders Fifth Edition (DSM-V) as a gradual cognitive decline following the established onset of Parkinson's Disease. The development of dementia in PD occurs in a spectrum; mild cognitive impairment in PD (PD-MCI) predicts the development of dementia. PD-MCI, termed mild NCD due to PD accounts for an estimated 27% of patients with PD according to the DSM-V.

Falls are a significant issue in PD patients. Up to 70% of PD patients fall annually, and according to the National Parkinson's Foundation, 38% of people with PD experience recurrent falls. In PDD and PD-MCI patients, falls are a particular problem and are closely associated with the severity of cognitive dysfunction. PD patients with more severe cognitive impairment have been shown to fall more frequently relative to PD patients without impaired cognition. In addition, falls are a significant source of burden to the healthcare system more broadly: over 700,000 patients a year are hospitalized because of a fall-related injury, accounting for direct medical costs of $34 billion in 2013 alone.

Deficits in cognition are closely linked to fall risk in patients with PDD, given the role of attention in walking. PD patients who are unable to perform another cognitive task while walking have increased gait variability and a significant increase in the incidence of falls. Deficits in dual task testing, a popular method of testing attention, have been shown to increase risk for gait abnormalities and increased recurrent falls. The association between attentional deficits and fall risk holds true even when controlling for severity of motor symptoms in PD. There is growing evidence that cholinergic deficits play a fundamental role in driving cognitive and attentional decline in PDD, given the role that acetylcholine plays in regulating attention via projections from the nucleus basalis of Meynert (nBM) to the dorsolateral prefrontal cortex. Hence, attentional deficits due to cholinergic losses in the nBM may be responsible for falls in patients with PD.

In addition to cholinergic degeneration in the nBM that drives attentional deficits, PD patients also undergo early degeneration of the pedunculopontine nucleus (PPN). The PPN provides cholinergic input to the thalamus, cerebellum, brainstem, striatum, and spinal cord and plays a critical role in the modulation of gait and locomotion. Furthermore, the PPN is functionally and anatomically located in the mesencephalic locomotor region (MLR) which is activated during gait. Indeed, bilateral lesioning of the cholinergic section of the PPN induces gait and postural deficits in monkeys. Importantly, these lesioned monkeys do not display deficits in global motor activity that are seen in dopaminergic lesioned monkeys, suggesting that lesioning of the PPN alone can induce deficits in gait and posture. Taken together, cholinergic loss of the nBM and PPN in PD, and to a greater extent in PDD, lead to gait disturbances, attentional deficits, and increased fall risk independent of dopaminergic pathology.

Anticholinergic medications are known to increase the risk of falls in the elderly, an observation that provides further evidence of the role of cholinergic deficits in driving the risk of falls in PD. Moreover, cholinesterase inhibitors have been shown to lower the risk of falls in PD. In a randomized, double-blind, placebo-controlled, crossover study of donepezil versus placebo in PD patients experiencing frequent falls, subjects reported nearly half as many falls while on donepezil compared to while on placebo (0.25 falls/day vs. 0.13 falls/day, p<0.05). Furthermore, in a 12 month double-blind, placebo-controlled study of 176 patients with either PD, PD-MCI, or PDD randomized to receive either rivastigmine or placebo, treatment with rivastigmine lowered the incidence of falls by 50% relative to baseline (p<0.01) while the placebo group had a 12% increase in falls/year relative to baseline. Drugs that increase the concentration of acetylcholine in the brain may therefore present promising therapeutic potential to reduce the risk of falls in PD, particularly in PD-MCI and PDD populations who are at highest risk.

Recently, adjunctive therapy with 5-HT6 antagonists on top of stable donepezil therapy have demonstrated reductions in the incidence of falls in patients with Alzheimer's disease. 5-HT6 antagonists work by selectively promoting the release of acetylcholine in regions of the brain related to cognition and function. In a 48-week double-blind placebo-controlled randomized study in mild-to-moderate Alzheimer's disease, 2% of subjects who received intepirdine on top of donepezil experienced falls, as compared to 6% of subjects who received donepezil alone. In addition, in a Phase 2 double-blind placebo-controlled randomized study of idalopirdine in moderate Alzheimer's disease, 2% of subjects who received idalopirdine on top of donepezil experienced falls, compared to 6% of subjects who received donepezil alone. Based on its mechanism of action as a 5-HT6 antagonist and its effects on reducing the incidence of falls in Alzheimer's disease, intepirdine may have the potential to reduce the risk of falls in patients with PDD and PD-MCI.

In addition, intepirdine may have the potential to improve the balance, gait, or both in patients with PDD and PD-MCI, as well. Although degradations in balance and increases in gait abnormalities are closely related to increases in recurrent falls, balance and gait issues often arise first. Patients in the earlier stages of PDD and PD-MCI might display balance and gait abnormalities that they are able to compensate for, because, for example, the deficits in cognition have not reached a point where falls are a likely result, the balance and gait abnormalities are minor enough that compensation is easier for the patient, or a combination thereof. Treating patients in the earlier stages of PDD and PD-MCI to improve their balance, gait or both may also allow, for example, for lower or more convenient dosages of intepirdine compared to patient in later stages of the disease where falls are already prevalent.

In some embodiments, a subject with a neurodegenerative disease has degradations in balance, gait or both. In some embodiments, the subject has a slow gait speed. In some embodiments, the subject has a slow gait speed of less than 89 cm/s. In some embodiments, the subject has a slow gait speed of less than 100 cm/s. In some embodiments, the subject exhibits Postural Instability and Gait Difficulty (PIGD) subtype assessed by the MDS-UPDRS. In some embodiments, the subject exhibits Postural Instability and Gait Difficulty (PIGD) subtype assessed by the MDS-UPDRS prior to administering to said subject a therapeutically effective amount of intepirdine or pharmaceutically acceptable salts, hydrates or solvates thereof. In some embodiments, the subject is aged between about 50 and about 95 years of age.

In some embodiments, a subject with a neurodegenerative disease has degradations in balance, gait, or both, but has not experienced an increase in falls. For example, a subject in the earlier stages of PDD has difficulties with walking, but has not yet experienced an increase in the rate of falling due to the effects of the disease. In some embodiments, the subject is determined to not have had an increase in falls as assessed by history gathered by a clinical investigator or physician before the administration of intepirdine. In some embodiments, the subject has not experienced an increase in falls within the last month. In some embodiments, the subject has not experienced an increase in falls within the last two months. In some embodiments, the subject has not experienced an increase in falls within the last three months. In some embodiments, the subject has not experienced an increase in falls within the last six months. In some embodiments, the subject has not experienced an increase in falls within the last twelve months. In some embodiments, the subject has experienced less than one fall per week prior to administration of intepirdine. In some embodiments, the subject has experienced less than 2 falls per month prior to administration of intepirdine. In some embodiments, the subject has experienced less than one fall per month prior to administration of intepirdine. In some embodiments, the subject has not experienced any falls prior to administration of intepirdine. In some embodiments, the subject exhibits Postural Instability and Gait Difficulty (PIGD) subtype assessed by the MDS-UPDRS, but has fallen less than once per week prior to administration of intepirdine. In some embodiments, the subject exhibits Postural Instability and Gait Difficulty (PIGD) subtype assessed by the MDS-UPDRS, but has fallen less than twice per month prior to administration of intepirdine. In some embodiments, the subject exhibits Postural Instability and Gait Difficulty (PIGD) subtype assessed by the MDS-UPDRS, but has fallen less than once per month prior to administration of intepirdine. In some embodiments, the subject exhibits Postural Instability and Gait Difficulty (PIGD) subtype assessed by the MDS-UPDRS, but has not experienced any falls prior to administration of intepirdine.

In some embodiments, a subject with a neurodegenerative disease has degradations in balance, gait, or both, and has experienced an increase in falls. For example, a subject in the later stages of PDD has difficulties with walking, and has also experienced an increase in the rate of falling due to the effects of the disease. In some embodiments, the subject is determined to have had an increase in falls as assessed by history gathered by a clinical investigator or physician before the administration of intepirdine. In some embodiments, the subject has experienced falls at least three days per week prior to administration of intepirdine. In some embodiments, the subject has experienced at least one fall over the past year prior to administration of intepirdine. In some embodiments, the subject has experienced at least three falls in the six months preceding administering to said subject a therapeutically effective amount of intepirdine. In some embodiments, the subject has a history of frequent falls. In some embodiments, the subject is experiencing falls at least three days per week for a period of at least two weeks prior to administration of intepirdine.

In some embodiments, a subject with a neurodegenerative disease is given a Mini-Mental State Evaluation (MMSE) battery to determine the severity and progression of the disease. The MMSE (Folstein et al., 1975) consists of 11 tests of orientation, memory (recent and immediate), concentration, language, and praxis. Scores range from 0 to 30, with lower scores indicating greater cognitive impairment. It is based on the performance of the subject and takes approximately 5 to 10 minutes to administer. In some embodiments, a subject in the earlier stages of a neurodegenerative disease has a MMSE score between 10 and 30. In some embodiments, a subject in the earlier stages of a neurodegenerative disease has a MMSE score between 12 and 30. In some embodiments, a subject in the earlier stages of a neurodegenerative disease has a MMSE score between 14 and 30. In some embodiments, a subject in the earlier stages of a neurodegenerative disease has a MMSE score between 16 and 30. In some embodiments, a subject in the earlier stages of a neurodegenerative disease has a MMSE score between 18 and 30. In some embodiments, a subject in the earlier stages of a neurodegenerative disease has a MMSE score between 20 and 30. In some embodiments, a subject in the earlier stages of a neurodegenerative disease has a MMSE score between 22 and 30. In some embodiments, a subject in the earlier stages of a neurodegenerative disease has a MMSE score between 24 and 30. In some embodiments, a subject in the earlier stages of a neurodegenerative disease has a MMSE score between 10 and 28. In some embodiments, a subject in the earlier stages of a neurodegenerative disease has a MMSE score between 10 and 26. In some embodiments, a subject in the earlier stages of a neurodegenerative disease has a MMSE score between 10 and 24. In some embodiments, a subject in the earlier stages of a neurodegenerative disease has a MMSE score between 10 and 22. In some embodiments, a subject in the earlier stages of a neurodegenerative disease has a MMSE score between 10 and 20. In some embodiments, a subject in the earlier stages of a neurodegenerative disease has a MMSE score between 18 and 28.

In some embodiments, administration of a therapeutically effective amount of intepirdine, or pharmaceutically acceptable salts, hydrates or solvates thereof, to a subject with a neurodegenerative disease with degradations in balance, gait, or both, results in an improvement in balance, gait speed, functional mobility, quantitative measures of balance and gait, gait variability, freezing of gait, balance confidence, movement and balance, smell, attention and executive function, postural instability and gait difficulty, quality of life, dynamic gait and balance, orthostatic hypotension, or any combination thereof.

In some embodiments, administration of intepirdine, or pharmaceutically acceptable salts, hydrates or solvates thereof, results in a change in quantitative measures of balance and gait after treatment. In some embodiments, administration of intepirdine or pharmaceutically acceptable salts, hydrates or solvates thereof results in a change in quantitative measures of balance and gait after about 12 weeks of treatment. In some embodiments, administration of intepirdine or pharmaceutically acceptable salts, hydrates or solvates thereof results in a change in quantitative measures of balance and gait after about 4 weeks of treatment. In some embodiments, administration of intepirdine or pharmaceutically acceptable salts, hydrates or solvates thereof results in a change in quantitative measures of balance and gait after about 4 to about 12 weeks of treatment. In some embodiments, administration of intepirdine or pharmaceutically acceptable salts, hydrates or solvates thereof results in a change in quantitative measures of balance and gait after about 2 weeks of treatment. In some embodiments, administration of intepirdine, or pharmaceutically acceptable salts, hydrates or solvates thereof, results in an improvement in quantitative measures of balance and gait after treatment. In some embodiments, administration of intepirdine or pharmaceutically acceptable salts, hydrates or solvates thereof results in an improvement in quantitative measures of balance and gait after about 12 weeks of treatment. In some embodiments, administration of intepirdine or pharmaceutically acceptable salts, hydrates or solvates thereof results in an improvement in quantitative measures of balance and gait after about 4 weeks of treatment. In some embodiments, administration of intepirdine or pharmaceutically acceptable salts, hydrates or solvates thereof results in an improvement in quantitative measures of balance and gait after about 4 to about 12 weeks of treatment. In some embodiments, administration of intepirdine or pharmaceutically acceptable salts, hydrates or solvates thereof results in an improvement in quantitative measures of balance and gait after about 2 weeks of treatment. In some embodiments, these quantitative measures are gait analysis under single and dual-task conditions, accelerometer testing, mini-BESTest evaluation, BERG balance testing, or any combination thereof. In some embodiments, the subject has the ability to stand for a longer period of time after the administration of intepirdine or pharmaceutically acceptable salts, hydrates or solvates thereof. In some embodiments, the balance and gait are measured by mini-BESTest battery total score, or individual item scores calculated by Opal APDM sensors during performance of the mini-BESTest battery. In some embodiments, balance and gait are changed from baseline in the mini-BESTest total score at the end of the treatment period. In some embodiments, balance and gait are changed from baseline in the individual item scores calculated by Opal APDM sensors during performance of the mini-BESTest battery at the end of the treatment period.

In some embodiments, administration of a therapeutically effective amount of intepirdine, or pharmaceutically acceptable salts, hydrates or solvates thereof, results in a change in dynamic gait and balance as measured by a change from baseline in measures of dynamic gait and balance assessed by data reduction and factor analysis using APDM Opal sensors during a mini-BESTest. In some embodiments, administration of a therapeutically effective amount of intepirdine, or pharmaceutically acceptable salts, hydrates or solvates thereof, results in a change in dynamic gait and balance as measured by a change from baseline in measures of dynamic gait and balance assessed by data reduction and factor analysis using APDM Opal sensors during a mini-BESTest after about 12 weeks of treatment. In some embodiments, administration of a therapeutically effective amount of intepirdine, or pharmaceutically acceptable salts, hydrates or solvates thereof, results in a change in dynamic gait and balance as measured by a change from baseline in measures of dynamic gait and balance assessed by data reduction and factor analysis using APDM Opal sensors during a mini-BESTest after about 2 weeks of treatment. In some embodiments, administration of a therapeutically effective amount of intepirdine, or pharmaceutically acceptable salts, hydrates or solvates thereof, results in an improvement in dynamic gait and balance as measured by a change from baseline in measures of dynamic gait and balance assessed by data reduction and factor analysis using APDM Opal sensors during a mini-BESTest. In some embodiments, administration of a therapeutically effective amount of intepirdine, or pharmaceutically acceptable salts, hydrates or solvates thereof, results in an improvement in dynamic gait and balance as measured by a change from baseline in measures of dynamic gait and balance assessed by data reduction and factor analysis using APDM Opal sensors during a mini-BESTest after about 12 weeks of treatment. In some embodiments, administration of a therapeutically effective amount of intepirdine, or pharmaceutically acceptable salts, hydrates or solvates thereof, results in an improvement in dynamic gait and balance as measured by a change from baseline in measures of dynamic gait and balance assessed by data reduction and factor analysis using APDM Opal sensors during a mini-BESTest after about 2 weeks of treatment.

In some embodiments, administration of a therapeutically effective amount of intepirdine, or pharmaceutically acceptable salts, hydrates or solvates thereof, results in an improvement in balance. In some embodiments, administration of a therapeutically effective amount of intepirdine, or pharmaceutically acceptable salts, hydrates or solvates thereof, results in an improvement in balance after about 12 weeks of treatment. In some embodiments, administration of a therapeutically effective amount of intepirdine, or pharmaceutically acceptable salts, hydrates or solvates thereof, results in an improvement in balance after about 2 weeks of treatment. In some embodiments, administration of a therapeutically effective amount of intepirdine, or pharmaceutically acceptable salts, hydrates or solvates thereof, results in a change in balance. In some embodiments, administration of a therapeutically effective amount of intepirdine, or pharmaceutically acceptable salts, hydrates or solvates thereof, results in a change in balance after about 12 weeks of treatment. In some embodiments, administration of a therapeutically effective amount of intepirdine, or pharmaceutically acceptable salts, hydrates or solvates thereof, results in a change in balance after about 2 weeks of treatment. In some embodiments, an improvement or a change in balance is assessed by a change from baseline in the root mean square (RMS) of mediolateral sway, measured using Opal APDM sensors during the mini-BESTest evaluation.

In some embodiments, administration of a therapeutically effective amount of intepirdine, or pharmaceutically acceptable salts, hydrates or solvates thereof, results in the improvement of balance in the elderly subject. In some embodiments, administration of a therapeutically effective amount of intepirdine, or pharmaceutically acceptable salts, hydrates or solvates thereof, results in the improvement of balance in subjects with dementia. In some embodiments, administration of a therapeutically effective amount of intepirdine, or pharmaceutically acceptable salts, hydrates or solvates thereof, results in the improvement of balance in subjects with Parkinson's disease. In some embodiments, administration of a therapeutically effective amount of intepirdine, or pharmaceutically acceptable salts, hydrates or solvates thereof, results in the improvement of balance in subjects with PD dementia. In some embodiments, administration of a therapeutically effective amount of intepirdine, or pharmaceutically acceptable salts, hydrates or solvates thereof, results in the improvement of balance in subjects with mild cognitive impairment in PD. In some embodiments, administration of a therapeutically effective amount of intepirdine, or pharmaceutically acceptable salts, hydrates or solvates thereof, results in the improvement of balance in subjects with Alzheimer's disease. In some embodiments, administration of a therapeutically effective amount of intepirdine, or pharmaceutically acceptable salts, hydrates or solvates thereof, results in the improvement of balance in subjects with dementia with Lewy Bodies. In some embodiments, administration of a therapeutically effective amount of intepirdine, or pharmaceutically acceptable salts, hydrates or solvates thereof, results in the improvement of balance in subjects with vascular dementia. In some embodiments, administration of a therapeutically effective amount of intepirdine, or pharmaceutically acceptable salts, hydrates or solvates thereof, results in the improvement of balance in subjects with mixed Alzheimer's disease and cardiovascular disease. In some embodiments, administration of a therapeutically effective amount of intepirdine, or pharmaceutically acceptable salts, hydrates or solvates thereof, results in the improvement of balance in subjects with Huntington's disease. In some embodiments, administration of a therapeutically effective amount of intepirdine, or pharmaceutically acceptable salts, hydrates or solvates thereof, results in the improvement of balance in subjects with Levodopa-induced dyskinesia in PD. In some embodiments, administration of a therapeutically effective amount of intepirdine, or pharmaceutically acceptable salts, hydrates or solvates thereof, results in the improvement of balance in subjects with orthostatic hypotension in PD. In some embodiments, administration of a therapeutically effective amount of intepirdine, or pharmaceutically acceptable salts, hydrates or solvates thereof, results in the improvement of balance in subjects with multiple sclerosis.

In some embodiments, administration of a therapeutically effective amount of intepirdine, or pharmaceutically acceptable salts, hydrates or solvates thereof, results in a change in balance confidence as measured by change from baseline in the Activities-Specific Balance Confidence (ABC) scale. In some embodiments, administration of a therapeutically effective amount of intepirdine, or pharmaceutically acceptable salts, hydrates or solvates thereof, results in a change in balance confidence as measured by change from baseline in the Activities-Specific Balance Confidence (ABC) scale after about 12 weeks of treatment. In some embodiments, administration of a therapeutically effective amount of intepirdine, or pharmaceutically acceptable salts, hydrates or solvates thereof, results in a change in balance confidence as measured by change from baseline in the Activities-Specific Balance Confidence (ABC) scale after about 2 weeks of treatment. In some embodiments, administration of a therapeutically effective amount of intepirdine, or pharmaceutically acceptable salts, hydrates or solvates thereof, results in an improvement in balance confidence as measured by change from baseline in the Activities-Specific Balance Confidence (ABC) scale. In some embodiments, administration of a therapeutically effective amount of intepirdine, or pharmaceutically acceptable salts, hydrates or solvates thereof, results in an improvement in balance confidence as measured by change from baseline in the Activities-Specific Balance Confidence (ABC) scale after about 12 weeks of treatment. In some embodiments, administration of a therapeutically effective amount of intepirdine, or pharmaceutically acceptable salts, hydrates or solvates thereof, results in an improvement in balance confidence as measured by change from baseline in the Activities-Specific Balance Confidence (ABC) scale after about 2 weeks of treatment. In some embodiments, balance confidence is changed from baseline in the ABC scale score at the end of the treatment period.

In some embodiments, administration of a therapeutically effective amount of intepirdine, or pharmaceutically acceptable salts, hydrates or solvates thereof, results in a change in balance confidence as measured by the Caregiver-Adapted Version of the Falls Efficacy Scale—International version. In some embodiments, administration of a therapeutically effective amount of intepirdine, or pharmaceutically acceptable salts, hydrates or solvates thereof, results in a change in balance confidence as measured by the Caregiver-Adapted Version of the Falls Efficacy Scale—International after about 12 weeks of treatment. In some embodiments, administration of a therapeutically effective amount of intepirdine, or pharmaceutically acceptable salts, hydrates or solvates thereof, results in a change in balance confidence as measured by the Caregiver-Adapted Version of the Falls Efficacy Scale—International after about 2 weeks of treatment. In some embodiments, administration of a therapeutically effective amount of intepirdine, or pharmaceutically acceptable salts, hydrates or solvates thereof, results in an improvement in balance confidence as measured by the Caregiver-Adapted Version of the Falls Efficacy Scale—International. In some embodiments, administration of a therapeutically effective amount of intepirdine, or pharmaceutically acceptable salts, hydrates or solvates thereof, results in an improvement in balance confidence as measured by the Caregiver-Adapted Version of the Falls Efficacy Scale—International after about 12 weeks of treatment. In some embodiments, administration of a therapeutically effective amount of intepirdine, or pharmaceutically acceptable salts, hydrates or solvates thereof, results in an improvement in balance confidence as measured by change the Caregiver-Adapted Version of the Falls Efficacy Scale—International after about 2 weeks of treatment. In some embodiments, balance confidence is changed from the Caregiver-Adapted Version of the Falls Efficacy Scale—International at the end of the treatment period.

In some embodiments, administration of a therapeutically effective amount of intepirdine, or pharmaceutically acceptable salts, hydrates or solvates thereof, results in a change in movement and balance as measured by the subjects' Motor Disorder Society Unified Parkinson's Disease Rating Scale (MDS-UPDRS), MDS-UPDRS Part III sub-score, MDS-UPDRS PIGD score, or any combination thereof. In some embodiments, administration of a therapeutically effective amount of intepirdine, or pharmaceutically acceptable salts, hydrates or solvates thereof, results in a change in movement and balance as measured by the subjects' Motor Disorder Society Unified Parkinson's Disease Rating Scale (MDS-UPDRS), MDS-UPDRS Part III sub-score, MDS-UPDRS PIGD score, or any combination thereof after about 12 weeks of treatment. In some embodiments, administration of a therapeutically effective amount of intepirdine, or pharmaceutically acceptable salts, hydrates or solvates thereof, results in a change in movement and balance as measured by the subjects' Motor Disorder Society Unified Parkinson's Disease Rating Scale (MDS-UPDRS), MDS-UPDRS Part III sub-score, MDS-UPDRS PIGD score, or any combination thereof after about 2 weeks of treatment. In some embodiments, administration of a therapeutically effective amount of intepirdine, or pharmaceutically acceptable salts, hydrates or solvates thereof, results in an improvement in movement and balance as measured by the subjects' Motor Disorder Society Unified Parkinson's Disease Rating Scale (MDS-UPDRS), MDS-UPDRS Part III sub-score, MDS-UPDRS PIGD score, or any combination thereof. In some embodiments, administration of a therapeutically effective amount of intepirdine, or pharmaceutically acceptable salts, hydrates or solvates thereof, results in an improvement in movement and balance as measured by the subjects' Motor Disorder Society Unified Parkinson's Disease Rating Scale (MDS-UPDRS), MDS-UPDRS Part III sub-score, MDS-UPDRS PIGD score, or any combination thereof after about 12 weeks of treatment. In some embodiments, administration of a therapeutically effective amount of intepirdine, or pharmaceutically acceptable salts, hydrates or solvates thereof, results in an improvement in movement and balance as measured by the subjects' Motor Disorder Society Unified Parkinson's Disease Rating Scale (MDS-UPDRS), MDS-UPDRS Part III sub-score, MDS-UPDRS PIGD score, or any combination thereof after about 2 weeks of treatment. In some embodiments, movement and balance is changed from baseline in MDS-UPDRS Part III sub-score at the end of the treatment period.

In some embodiments, administration of a therapeutically effective amount of intepirdine, or pharmaceutically acceptable salts, hydrates or solvates thereof, results in an improvement in gait. In some embodiments, administration of a therapeutically effective amount of intepirdine, or pharmaceutically acceptable salts, hydrates or solvates thereof, results in an improvement in gait after about 12 weeks of treatment. In some embodiments, administration of a therapeutically effective amount of intepirdine, or pharmaceutically acceptable salts, hydrates or solvates thereof, results in an improvement in gait after about 2 weeks of treatment. In some embodiments, administration of a therapeutically effective amount of intepirdine, or pharmaceutically acceptable salts, hydrates or solvates thereof, results in a change in gait. In some embodiments, administration of a therapeutically effective amount of intepirdine, or pharmaceutically acceptable salts, hydrates or solvates thereof, results in a change in gait after about 12 weeks of treatment. In some embodiments, administration of a therapeutically effective amount of intepirdine, or pharmaceutically acceptable salts, hydrates or solvates thereof, results in a change in gait after about 2 weeks of treatment. In some embodiments, an improvement or change in gait is assessed by a change from baseline in step time variability, stride time variability, stride length variability, step length variability, gait speed or any combination thereof, under normal and dual-task walking conditions measured on a GAITRite electronic walkway system.

In some embodiments, administration of a therapeutically effective amount of intepirdine, or pharmaceutically acceptable salts, hydrates or solvates thereof, results in the improvement of gait in the elderly subject. In some embodiments, administration of a therapeutically effective amount of intepirdine, or pharmaceutically acceptable salts, hydrates or solvates thereof, results in the improvement of gait in subjects with dementia. In some embodiments, administration of a therapeutically effective amount of intepirdine, or pharmaceutically acceptable salts, hydrates or solvates thereof, results in the improvement of gait in subjects with Parkinson's disease. In some embodiments, administration of a therapeutically effective amount of intepirdine, or pharmaceutically acceptable salts, hydrates or solvates thereof, results in the improvement of gait in subjects with PD dementia. In some embodiments, administration of a therapeutically effective amount of intepirdine, or pharmaceutically acceptable salts, hydrates or solvates thereof, results in the improvement of gait in subjects with mild cognitive impairment in PD. In some embodiments, administration of a therapeutically effective amount of intepirdine, or pharmaceutically acceptable salts, hydrates or solvates thereof, results in the improvement of gait in subjects with Alzheimer's disease. In some embodiments, administration of a therapeutically effective amount of intepirdine, or pharmaceutically acceptable salts, hydrates or solvates thereof, results in the improvement of gait in subjects with dementia with Lewy Bodies. In some embodiments, administration of a therapeutically effective amount of intepirdine, or pharmaceutically acceptable salts, hydrates or solvates thereof, results in the improvement of gait in subjects with vascular dementia. In some embodiments, administration of a therapeutically effective amount of intepirdine, or pharmaceutically acceptable salts, hydrates or solvates thereof, results in the improvement of gait in subjects with mixed Alzheimer's disease and cardiovascular disease. In some embodiments, administration of a therapeutically effective amount of intepirdine, or pharmaceutically acceptable salts, hydrates or solvates thereof, results in the improvement of gait in subjects with Huntington's disease. In some embodiments, administration of a therapeutically effective amount of intepirdine, or pharmaceutically acceptable salts, hydrates or solvates thereof, results in the improvement of gait in subjects with Levodopa-induced dyskinesia in PD. In some embodiments, administration of a therapeutically effective amount of intepirdine, or pharmaceutically acceptable salts, hydrates or solvates thereof, results in the improvement of gait in subjects with orthostatic hypotension in PD. In some embodiments, administration of a therapeutically effective amount of intepirdine, or pharmaceutically acceptable salts, hydrates or solvates thereof, results in the improvement of gait in subjects with multiple sclerosis.

In some embodiments, administration of a therapeutically effective amount of intepirdine, or pharmaceutically acceptable salts, hydrates or solvates thereof, results in an improvement in gait speed. In some embodiments, administration of a therapeutically effective amount of intepirdine, or pharmaceutically acceptable salts, hydrates or solvates thereof, results in an improvement in gait speed after about 12 weeks of treatment. In some embodiments, administration of a therapeutically effective amount of intepirdine, or pharmaceutically acceptable salts, hydrates or solvates thereof, results in an improvement in gait speed after about 2 weeks of treatment. In some embodiments, administration of a therapeutically effective amount of intepirdine, or pharmaceutically acceptable salts, hydrates or solvates thereof, results in a change in gait speed. In some embodiments, administration of a therapeutically effective amount of intepirdine, or pharmaceutically acceptable salts, hydrates or solvates thereof, results in a change in gait speed after about 12 weeks of treatment. In some embodiments, administration of a therapeutically effective amount of intepirdine, or pharmaceutically acceptable salts, hydrates or solvates thereof, results in a change in gait speed after about 2 weeks of treatment. In some embodiments, an improvement or change in gait speed is assessed by quantitatively measuring the functional mobility, evaluated under normal and dual-task walking conditions on an electronic walkway system. In some embodiments, gait speed is changed from baseline under normal and dual-task walking conditions measured on an electronic walkway system at the end of the treatment period.

In some embodiments, administration of a therapeutically effective amount of intepirdine, or pharmaceutically acceptable salts, hydrates or solvates thereof, results in an improvement in gait variability. In some embodiments, administration of a therapeutically effective amount of intepirdine, or pharmaceutically acceptable salts, hydrates or solvates thereof, results in an improvement in gait variability after about 12 weeks of treatment. In some embodiments, administration of a therapeutically effective amount of intepirdine, or pharmaceutically acceptable salts, hydrates or solvates thereof, results in an improvement in gait variability after about 2 weeks of treatment. In some embodiments, administration of a therapeutically effective amount of intepirdine, or pharmaceutically acceptable salts, hydrates or solvates thereof, results in a change in gait variability. In some embodiments, administration of a therapeutically effective amount of intepirdine, or pharmaceutically acceptable salts, hydrates or solvates thereof, results in a change in gait variability after about 12 weeks of treatment. In some embodiments, administration of a therapeutically effective amount of intepirdine, or pharmaceutically acceptable salts, hydrates or solvates thereof, results in a change in gait variability after about 2 weeks of treatment. In some embodiments, an improvement or change in gait variability is evaluated under normal and dual-task walking conditions on an electronic walkway system. In some embodiments, gait variability is changed from baseline under normal and dual-task walking conditions measured on an electronic walkway system at the end of the treatment period.

In some embodiments, administration of a therapeutically effective amount of intepirdine, or pharmaceutically acceptable salts, hydrates or solvates thereof, results in a change in freezing of gait (FOG). In some embodiments, administration of a therapeutically effective amount of intepirdine, or pharmaceutically acceptable salts, hydrates or solvates thereof, results in an improvement in freezing of gait (FOG). In some embodiments, administration of a therapeutically effective amount of intepirdine, or pharmaceutically acceptable salts, hydrates or solvates thereof, results in a decrease in freezing of gait (FOG). In some embodiments, administration of a therapeutically effective amount of intepirdine, or pharmaceutically acceptable salts, hydrates or solvates thereof, results in a change in freezing of gait (FOG) after about 12 weeks of treatment. In some embodiments, administration of a therapeutically effective amount of intepirdine, or pharmaceutically acceptable salts, hydrates or solvates thereof, results in an improvement in freezing of gait (FOG) after about 12 weeks of treatment. In some embodiments, administration of a therapeutically effective amount of intepirdine, or pharmaceutically acceptable salts, hydrates or solvates thereof, results in a decrease in freezing of gait (FOG) after 12 weeks of treatment. In some embodiments, administration of a therapeutically effective amount of intepirdine, or pharmaceutically acceptable salts, hydrates or solvates thereof, results in a change in freezing of gait (FOG) after about 4 weeks of treatment. In some embodiments, administration of a therapeutically effective amount of intepirdine, or pharmaceutically acceptable salts, hydrates or solvates thereof, results in an improvement in freezing of gait (FOG) after about 4 weeks of treatment. In some embodiments, administration of a therapeutically effective amount of intepirdine, or pharmaceutically acceptable salts, hydrates or solvates thereof, results in a decrease in freezing of gait (FOG) after 4 weeks of treatment. In some embodiments, administration of a therapeutically effective amount of intepirdine, or pharmaceutically acceptable salts, hydrates or solvates thereof, results in a change in freezing of gait (FOG) after about 2 weeks of treatment. In some embodiments, administration of a therapeutically effective amount of intepirdine, or pharmaceutically acceptable salts, hydrates or solvates thereof, results in an improvement in freezing of gait (FOG) after about 2 weeks of treatment. In some embodiments, administration of a therapeutically effective amount of intepirdine, or pharmaceutically acceptable salts, hydrates or solvates thereof, results in a decrease in freezing of gait (FOG) after about 2 weeks of treatment. In some embodiments, administration of a therapeutically effective amount of intepirdine, or pharmaceutically acceptable salts, hydrates or solvates thereof, results in a change in freezing of gait (FOG) after about 4 to about 12 weeks of treatment. In some embodiments, administration of a therapeutically effective amount of intepirdine, or pharmaceutically acceptable salts, hydrates or solvates thereof, results in an improvement in freezing of gait (FOG) after about 4 to about 12 weeks of treatment. In some embodiments, administration of a therapeutically effective amount of intepirdine, or pharmaceutically acceptable salts, hydrates or solvates thereof, results in a decrease in freezing of gait (FOG) after about 4 to about 12 weeks of treatment. In some embodiments, freezing of gait (FOG) is measured by rapid 360-degree turns test, rapid full turns test, walking under normal and dual task trajectory, walking under normal and dual task conditions, change in baseline in Freezing ratio, measured using APDM Opal sensors, GAITRite analysis, and a combination thereof. In some embodiments, freezing of gait (FOG) is changed from baseline in the incidence of FOG episodes under rapid full turns test conditions and walking under normal and dual task trajectory at the end of the treatment period.

In some embodiments, administration of a therapeutically effective amount of intepirdine, or pharmaceutically acceptable salts, hydrates or solvates thereof, results in a change in Postural Instability and Gait Difficulty subscore (PIGD) of the UPDRS-III (items 27-30). In some embodiments, administration of a therapeutically effective amount of intepirdine, or pharmaceutically acceptable salts, hydrates or solvates thereof, results in an improvement in Postural Instability and Gait Difficulty subscore (PIGD) of the UPDRS-III (items 27-30). In some embodiments, administration of a therapeutically effective amount of intepirdine, or pharmaceutically acceptable salts, hydrates or solvates thereof, results in a change in Postural Instability and Gait Difficulty subscore (PIGD) of the UPDRS-III (items 27-30) after about 12 weeks of treatment. In some embodiments, administration of a therapeutically effective amount of intepirdine, or pharmaceutically acceptable salts, hydrates or solvates thereof, results in an improvement in Postural Instability and Gait Difficulty subscore (PIGD) of the UPDRS-III (items 27-30) after about 12 weeks of treatment. In some embodiments, administration of a therapeutically effective amount of intepirdine, or pharmaceutically acceptable salts, hydrates or solvates thereof, results in a change in Postural Instability and Gait Difficulty subscore (PIGD) of the UPDRS-III (items 27-30) after about 4 weeks of treatment. In some embodiments, administration of a therapeutically effective amount of intepirdine, or pharmaceutically acceptable salts, hydrates or solvates thereof, results in an improvement in Postural Instability and Gait Difficulty subscore (PIGD) of the UPDRS-III (items 27-30) after about 4 weeks of treatment. In some embodiments, administration of a therapeutically effective amount of intepirdine, or pharmaceutically acceptable salts, hydrates or solvates thereof, results in a change in Postural Instability and Gait Difficulty subscore (PIGD) of the UPDRS-III (items 27-30) after about 2 weeks of treatment. In some embodiments, administration of a therapeutically effective amount of intepirdine, or pharmaceutically acceptable salts, hydrates or solvates thereof, results in an improvement in Postural Instability and Gait Difficulty subscore (PIGD) of the UPDRS-III (items 27-30) after about 4 weeks of treatment. In some embodiments, administration of a therapeutically effective amount of intepirdine, or pharmaceutically acceptable salts, hydrates or solvates thereof, results in a change in Postural Instability and Gait Difficulty subscore (PIGD) of the UPDRS-III (items 27-30) after about 4 to about 12 weeks of treatment. In some embodiments, administration of a therapeutically effective amount of intepirdine, or pharmaceutically acceptable salts, hydrates or solvates thereof, results in an improvement in Postural Instability and Gait Difficulty subscore (PIGD) of the UPDRS-III (items 27-30) after about 4 to about 12 weeks of treatment.

In some embodiments, administration of intepirdine, or pharmaceutically acceptable salts, hydrates or solvates thereof, results in a change in attention after treatment as measured by the Cognitive Drug Research Power of Attention computerized test. In some embodiments, administration of intepirdine, or pharmaceutically acceptable salts, hydrates or solvates thereof, results in a change in attention after about 12 weeks of treatment as measured by the Cognitive Drug Research Power of Attention computerized test. In some embodiments, administration of intepirdine, or pharmaceutically acceptable salts, hydrates or solvates thereof, results in a change in attention after about 4 weeks of treatment as measured by the Cognitive Drug Research Power of Attention computerized test. In some embodiments, administration of intepirdine, or pharmaceutically acceptable salts, hydrates or solvates thereof, results in a change in attention after about 4 to about 12 weeks of treatment as measured by the Cognitive Drug Research Power of Attention computerized test. In some embodiments, administration of intepirdine, or pharmaceutically acceptable salts, hydrates or solvates thereof, results in a change in attention after about 2 weeks of treatment as measured by the Cognitive Drug Research Power of Attention computerized test. In some embodiments, administration of intepirdine, or pharmaceutically acceptable salts, hydrates or solvates thereof, results in an improvement in attention after treatment as measured by the Cognitive Drug Research Power of Attention computerized test. In some embodiments, administration of intepirdine, or pharmaceutically acceptable salts, hydrates or solvates thereof, results in an improvement in attention after about 12 weeks of treatment as measured by the Cognitive Drug Research Power of Attention computerized test. In some embodiments, administration of intepirdine, or pharmaceutically acceptable salts, hydrates or solvates thereof, results in an improvement in attention after about 4 weeks of treatment as measured by the Cognitive Drug Research Power of Attention computerized test. In some embodiments, administration of intepirdine, or pharmaceutically acceptable salts, hydrates or solvates thereof, results in an improvement in attention after about 4 to about 12 weeks of treatment as measured by the Cognitive Drug Research Power of Attention computerized test. In some embodiments, administration of intepirdine, or pharmaceutically acceptable salts, hydrates or solvates thereof, results in an improvement in attention after about 2 weeks of treatment as measured by the Cognitive Drug Research Power of Attention computerized test.

In some embodiments, administration of a therapeutically effective amount of intepirdine, or pharmaceutically acceptable salts, hydrates or solvates thereof, results in an improvement in attention and executive function. In some embodiments, administration of a therapeutically effective amount of intepirdine, or pharmaceutically acceptable salts, hydrates or solvates thereof, results in a change in attention and executive function. In some embodiments, administration of a therapeutically effective amount of intepirdine, or pharmaceutically acceptable salts, hydrates or solvates thereof, results in an improvement in attention and executive function after about 12 weeks of treatment as measured by the Cognitive Drug Research Computerized Assessment System for Dementia. In some embodiments, administration of a therapeutically effective amount of intepirdine, or pharmaceutically acceptable salts, hydrates or solvates thereof, results in a change in attention and executive function after about 12 weeks of treatment as measured by the Cognitive Drug Research Computerized Assessment System for Dementia. In some embodiments, administration of a therapeutically effective amount of intepirdine, or pharmaceutically acceptable salts, hydrates or solvates thereof, results in an improvement in attention and executive function after about 4 weeks of treatment as measured by the Cognitive Drug Research Computerized Assessment System for Dementia. In some embodiments, administration of a therapeutically effective amount of intepirdine, or pharmaceutically acceptable salts, hydrates or solvates thereof, results in a change in attention and executive function after about 4 weeks of treatment as measured by the Cognitive Drug Research Computerized Assessment System for Dementia. In some embodiments, administration of a therapeutically effective amount of intepirdine, or pharmaceutically acceptable salts, hydrates or solvates thereof, results in an improvement in attention and executive function after about 2 weeks of treatment as measured by the Cognitive Drug Research Computerized Assessment System for Dementia. In some embodiments, administration of a therapeutically effective amount of intepirdine, or pharmaceutically acceptable salts, hydrates or solvates thereof, results in a change in attention and executive function after about 2 weeks of treatment as measured by the Cognitive Drug Research Computerized Assessment System for Dementia. In some embodiments, administration of a therapeutically effective amount of intepirdine, or pharmaceutically acceptable salts, hydrates or solvates thereof, results in an improvement in attention and executive function after about 4 to about 12 weeks of treatment as measured by the Cognitive Drug Research Computerized Assessment System for Dementia. In some embodiments, administration of a therapeutically effective amount of intepirdine, or pharmaceutically acceptable salts, hydrates or solvates thereof, results in a change in attention and executive function after about 4 to about 12 weeks of treatment as measured by the Cognitive Drug Research Computerized Assessment System for Dementia.

In some embodiments, administration of a therapeutically effective amount of intepirdine, or pharmaceutically acceptable salts, hydrates or solvates thereof, results in a change in attention and executive function after about 12 weeks of treatment as measured by change from baseline Power of Attention and the composite z-score combining the 7 COGDRAS-D domains. In some embodiments, administration of a therapeutically effective amount of intepirdine, or pharmaceutically acceptable salts, hydrates or solvates thereof, results in an improvement in attention and executive function after about 12 weeks of treatment as measured by change from baseline Power of Attention and the composite z-score combining the 7 COGDRAS-D domains. In some embodiments, administration of a therapeutically effective amount of intepirdine, or pharmaceutically acceptable salts, hydrates or solvates thereof, results in a change in attention and executive function after about 4 weeks of treatment as measured by change from baseline Power of Attention and the composite z-score combining the 7 COGDRAS-D domains. In some embodiments, administration of a therapeutically effective amount of intepirdine, or pharmaceutically acceptable salts, hydrates or solvates thereof, results in an improvement in attention and executive function after about 4 weeks of treatment as measured by change from baseline Power of Attention and the composite z-score combining the 7 COGDRAS-D domains. In some embodiments, administration of a therapeutically effective amount of intepirdine, or pharmaceutically acceptable salts, hydrates or solvates thereof, results in a change in attention and executive function after about 2 weeks of treatment as measured by change from baseline Power of Attention and the composite z-score combining the 7 COGDRAS-D domains. In some embodiments, administration of a therapeutically effective amount of intepirdine, or pharmaceutically acceptable salts, hydrates or solvates thereof, results in an improvement in attention and executive function after about 2 weeks of treatment as measured by change from baseline Power of Attention and the composite z-score combining the 7 COGDRAS-D domains. In some embodiments, administration of a therapeutically effective amount of intepirdine, or pharmaceutically acceptable salts, hydrates or solvates thereof, results in a change in attention and executive function after about 4 to about 12 weeks of treatment as measured by change from baseline Power of Attention and the composite z-score combining the 7 COGDRAS-D domains. In some embodiments, administration of a therapeutically effective amount of intepirdine, or pharmaceutically acceptable salts, hydrates or solvates thereof, results in an improvement in attention and executive function after about 4 to about 12 weeks of treatment as measured by change from baseline Power of Attention and the composite z-score combining the 7 COGDRAS-D domains.

In some embodiments, the subject exhibits a reduction in the incidence of falls, a reduction in the incidence of near falls, a delay in time to first fall, a delay in time to first near-fall, or any combination thereof after administration of a therapeutically effective amount of intepirdine, or pharmaceutically acceptable salts, hydrates or solvates thereof. In some embodiments, the subject exhibits a change in the incidence of falls, the incidence of near falls, a delay in time to first fall, a delay in time to first near-fall, or any combination thereof after administration of a therapeutically effective amount of intepirdine, or pharmaceutically acceptable salts, hydrates or solvates thereof. In some embodiments, the subject exhibits a decreased fear of falling, an increased confidence in balance, or both. In some embodiments, the subject exhibits a decreased fear of falling, an increased confidence in balance, or both after administration of a therapeutically effective amount of intepirdine, or pharmaceutically acceptable salts, hydrates or solvates thereof.

In some embodiments, administration of a therapeutically effective amount of intepirdine, or pharmaceutically acceptable salts, hydrates or solvates thereof, results in the prevention of falls. In some embodiments, administration of a therapeutically effective amount of intepirdine, or pharmaceutically acceptable salts, hydrates or solvates thereof, results in the prevention of falls in the elderly subject. In some embodiments, administration of a therapeutically effective amount of intepirdine, or pharmaceutically acceptable salts, hydrates or solvates thereof, results in the prevention of falls in subjects with dementia. In some embodiments, administration of a therapeutically effective amount of intepirdine, or pharmaceutically acceptable salts, hydrates or solvates thereof, results in the prevention of falls in subjects with Parkinson's disease. In some embodiments, administration of a therapeutically effective amount of intepirdine, or pharmaceutically acceptable salts, hydrates or solvates thereof, results in the prevention of falls in subjects with PD dementia. In some embodiments, administration of a therapeutically effective amount of intepirdine, or pharmaceutically acceptable salts, hydrates or solvates thereof, results in the prevention of falls in subjects with mild cognitive impairment in PD. In some embodiments, administration of a therapeutically effective amount of intepirdine, or pharmaceutically acceptable salts, hydrates or solvates thereof, results in the prevention of falls in subjects with Alzheimer's disease. In some embodiments, administration of a therapeutically effective amount of intepirdine, or pharmaceutically acceptable salts, hydrates or solvates thereof, results in the prevention of falls in subjects with dementia with Lewy Bodies. In some embodiments, administration of a therapeutically effective amount of intepirdine, or pharmaceutically acceptable salts, hydrates or solvates thereof, results in the prevention of falls in subjects with vascular dementia. In some embodiments, administration of a therapeutically effective amount of intepirdine, or pharmaceutically acceptable salts, hydrates or solvates thereof, results in the prevention of falls in subjects with mixed Alzheimer's disease and cardiovascular disease. In some embodiments, administration of a therapeutically effective amount of intepirdine, or pharmaceutically acceptable salts, hydrates or solvates thereof, results in the prevention of falls in subjects with Huntington's disease. In some embodiments, administration of a therapeutically effective amount of intepirdine, or pharmaceutically acceptable salts, hydrates or solvates thereof, results in the prevention of falls in subjects with Levodopa-induced dyskinesia in PD. In some embodiments, administration of a therapeutically effective amount of intepirdine, or pharmaceutically acceptable salts, hydrates or solvates thereof, results in the prevention of falls in subjects with orthostatic hypotension in PD. In some embodiments, administration of a therapeutically effective amount of intepirdine, or pharmaceutically acceptable salts, hydrates or solvates thereof, results in the prevention of falls in subjects with multiple sclerosis.

In some embodiments, administration of intepirdine, or pharmaceutically acceptable salts, hydrates or solvates thereof, results in a change in the Unified Parkinson's Disease Rating Scale and subscale. In some embodiments, administration of intepirdine or pharmaceutically acceptable salts, hydrates or solvates thereof results in an improvement in the Unified Parkinson's Disease Rating Scale and subscale.

In some embodiments, administration of intepirdine, or pharmaceutically acceptable salts, hydrates or solvates thereof, results in a change after treatment on a clinician global impression of change. In some embodiments, administration of intepirdine, or pharmaceutically acceptable salts, hydrates or solvates thereof, results in a change after about 12 weeks of treatment on a clinician global impression of change. In some embodiments, administration of intepirdine, or pharmaceutically acceptable salts, hydrates or solvates thereof, results in a change after about 4 weeks of treatment on a clinician global impression of change. In some embodiments, administration of intepirdine, or pharmaceutically acceptable salts, hydrates or solvates thereof, results in a change after about 2 weeks of treatment on a clinician global impression of change. In some embodiments, administration of intepirdine, or pharmaceutically acceptable salts, hydrates or solvates thereof, results in a change after about 4 to about 12 weeks of treatment on a clinician global impression of change. In some embodiments, administration of intepirdine, or pharmaceutically acceptable salts, hydrates or solvates thereof, results in an improvement after treatment on a clinician global impression of change. In some embodiments, administration of intepirdine, or pharmaceutically acceptable salts, hydrates or solvates thereof, results in an improvement after about 12 weeks of treatment on a clinician global impression of change. In some embodiments, administration of intepirdine, or pharmaceutically acceptable salts, hydrates or solvates thereof, results in an improvement after about 4 weeks of treatment on a clinician global impression of change. In some embodiments, administration of intepirdine, or pharmaceutically acceptable salts, hydrates or solvates thereof, results in an improvement after about 2 weeks of treatment on a clinician global impression of change. In some embodiments, administration of intepirdine, or pharmaceutically acceptable salts, hydrates or solvates thereof, results in an improvement after about 4 to about 12 weeks of treatment on a clinician global impression of change.

In some embodiments, administration of a therapeutically effective amount of intepirdine, or pharmaceutically acceptable salts, hydrates or solvates thereof, results in a change in smell, a proxy for cholinergic function, as measured by the University of Pennsylvania Smell Identification Test (UPSIT). In some embodiments, administration of a therapeutically effective amount of intepirdine, or pharmaceutically acceptable salts, hydrates or solvates thereof, results in a change in smell, a proxy for cholinergic function, as measured by the University of Pennsylvania Smell Identification Test (UPSIT) after about 12 weeks of treatment. In some embodiments, administration of a therapeutically effective amount of intepirdine, or pharmaceutically acceptable salts, hydrates or solvates thereof, results in a change in smell, a proxy for cholinergic function, as measured by the University of Pennsylvania Smell Identification Test (UPSIT) after about 2 weeks of treatment. In some embodiments, administration of a therapeutically effective amount of intepirdine, or pharmaceutically acceptable salts, hydrates or solvates thereof, results in an improvement in smell, a proxy for cholinergic function, as measured by the University of Pennsylvania Smell Identification Test (UPSIT). In some embodiments, administration of a therapeutically effective amount of intepirdine, or pharmaceutically acceptable salts, hydrates or solvates thereof, results in an improvement in smell, a proxy for cholinergic function, as measured by the University of Pennsylvania Smell Identification Test (UP SIT) after about 12 weeks of treatment. In some embodiments, administration of a therapeutically effective amount of intepirdine, or pharmaceutically acceptable salts, hydrates or solvates thereof, results in an improvement in smell, a proxy for cholinergic function, as measured by the University of Pennsylvania Smell Identification Test (UPSIT) after about 2 weeks of treatment. In some embodiments, smell is changed from baseline in UPSIT score at the end of the treatment period.

In some embodiments, administration of a therapeutically effective amount of intepirdine, or pharmaceutically acceptable salts, hydrates or solvates thereof, results in a change in quality of life as measured by change from baseline in the Parkinson's Disease Questionnaire-39 (PDQ-39). In some embodiments, administration of a therapeutically effective amount of intepirdine, or pharmaceutically acceptable salts, hydrates or solvates thereof, results in a change in quality of life as measured by change from baseline in the Parkinson's Disease Questionnaire-39 (PDQ-39) after about 12 weeks of treatment. In some embodiments, administration of a therapeutically effective amount of intepirdine, or pharmaceutically acceptable salts, hydrates or solvates thereof, results in a change in quality of life as measured by change from baseline in the Parkinson's Disease Questionnaire-39 (PDQ-39) after about 2 weeks of treatment. In some embodiments, administration of a therapeutically effective amount of intepirdine, or pharmaceutically acceptable salts, hydrates or solvates thereof, results in an improvement in quality of life as measured by change from baseline in the Parkinson's Disease Questionnaire-39 (PDQ-39). In some embodiments, administration of a therapeutically effective amount of intepirdine, or pharmaceutically acceptable salts, hydrates or solvates thereof, results in an improvement in quality of life as measured by change from baseline in the Parkinson's Disease Questionnaire-39 (PDQ-39) after about 12 weeks of treatment. In some embodiments, administration of a therapeutically effective amount of intepirdine, or pharmaceutically acceptable salts, hydrates or solvates thereof, results in an improvement in quality of life as measured by change from baseline in the Parkinson's Disease Questionnaire-39 (PDQ-39) after about 2 weeks of treatment.

In some embodiments, administration of a therapeutically effective amount of intepirdine, or pharmaceutically acceptable salts, hydrates or solvates thereof, results in the improvement of orthostatic hypotension. In some embodiments, administration of a therapeutically effective amount of intepirdine, or pharmaceutically acceptable salts, hydrates or solvates thereof, results in the improvement of orthostatic hypotension in the elderly subject. In some embodiments, administration of a therapeutically effective amount of intepirdine, or pharmaceutically acceptable salts, hydrates or solvates thereof, results in the improvement of orthostatic hypotension in subjects with dementia. In some embodiments, administration of a therapeutically effective amount of intepirdine, or pharmaceutically acceptable salts, hydrates or solvates thereof, results in the improvement of orthostatic hypotension in subjects with Parkinson's disease. In some embodiments, administration of a therapeutically effective amount of intepirdine, or pharmaceutically acceptable salts, hydrates or solvates thereof, results in the improvement of orthostatic hypotension in subjects with PD dementia. In some embodiments, administration of a therapeutically effective amount of intepirdine, or pharmaceutically acceptable salts, hydrates or solvates thereof, results in the improvement of orthostatic hypotension in subjects with mild cognitive impairment in PD. In some embodiments, administration of a therapeutically effective amount of intepirdine, or pharmaceutically acceptable salts, hydrates or solvates thereof, results in the improvement of orthostatic hypotension in subjects with Alzheimer's disease. In some embodiments, administration of a therapeutically effective amount of intepirdine, or pharmaceutically acceptable salts, hydrates or solvates thereof, results in the improvement of orthostatic hypotension in subjects with dementia with Lewy Bodies. In some embodiments, administration of a therapeutically effective amount of intepirdine, or pharmaceutically acceptable salts, hydrates or solvates thereof, results in the improvement of orthostatic hypotension in subjects with vascular dementia. In some embodiments, administration of a therapeutically effective amount of intepirdine, or pharmaceutically acceptable salts, hydrates or solvates thereof, results in the improvement of orthostatic hypotension in subjects with mixed Alzheimer's disease and cardiovascular disease. In some embodiments, administration of a therapeutically effective amount of intepirdine, or pharmaceutically acceptable salts, hydrates or solvates thereof, results in the improvement of orthostatic hypotension in subjects with Huntington's disease. In some embodiments, administration of a therapeutically effective amount of intepirdine, or pharmaceutically acceptable salts, hydrates or solvates thereof, results in the improvement of orthostatic hypotension in subjects with Levodopa-induced dyskinesia in PD. In some embodiments, administration of a therapeutically effective amount of intepirdine, or pharmaceutically acceptable salts, hydrates or solvates thereof, results in the improvement of orthostatic hypotension in subjects with orthostatic hypotension in PD. In some embodiments, administration of a therapeutically effective amount of intepirdine, or pharmaceutically acceptable salts, hydrates or solvates thereof, results in the improvement of orthostatic hypotension in subjects with multiple sclerosis.

It will be appreciated that reference to treatment is intended to include prophylaxis as well as the alleviation of established symptoms.

### Compositions/Formulations

In some embodiments, the compounds for use in the methods described herein may be formulated as pharmaceutical compositions. Pharmaceutical compositions of this disclosure may comprise the compounds described herein or a pharmaceutically acceptable salt thereof and a pharmaceutically acceptable carrier. Such compositions may optionally comprise an additional therapeutic agent.

The term “pharmaceutically acceptable carrier” refers to a non-toxic carrier that may be administered to a patient, together with a compound of this disclosure, and which does not destroy the pharmacological activity thereof. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Pharmaceutically acceptable carriers that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as α-tocopherol, polyethyleneglycol 1000 succinate, or other similar polymeric delivery matrices.

The compounds of this disclosure may be employed in a conventional manner for controlling the disease described herein, including, but not limited to, a neurodegenerative disease, and for treating diseases or reducing the advancement or severity of effects. Such methods of treatment, their dosage levels and requirements may be selected by those of ordinary skill in the art from available methods and techniques. For example, the compounds of this disclosure may be combined with a pharmaceutically acceptable adjuvant for administration to a patient suffering from a neurodegenerative disease in a pharmaceutically acceptable manner and in an amount effective to lessen the severity of that disease.

Alternatively, the compounds of this disclosure may be used in compositions and methods for treating or protecting individuals against the diseases described herein, including but not limited to a Parkinson's disease, Parkinson's disease dementia, or any combination thereof, over extended periods of time. The compounds may be employed in such compositions either alone or together with other compounds of this disclosure in a manner consistent with the conventional utilization of such compounds in pharmaceutical compositions and in some embodiments can be administered in prophylactically effective amounts to protect individuals over an extended period of time against the diseases described herein, including, but not limited to, Parkinson's disease, Parkinson's disease dementia, or any combination thereof.

As used herein, the terms “combination,” “combined,” and related terms refer to the simultaneous or sequential administration of therapeutic agents in accordance with this disclosure. For example, a described compound may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients (excipients). Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or non-aqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of this disclosure may also be administered in the form of suppositories for rectal administration. These compositions can be prepared by mixing a compound of this disclosure with a suitable non-irritating excipient, which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this disclosure may also be topically applied to the lower intestinal tract by rectal suppository formulation or in a suitable enema formulation. Topically-administered transdermal patches are also included in this disclosure.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Transdermal patches have the added advantage of providing controlled delivery of a compound of the present disclosure to the body. Dissolving or dispersing the compound in the proper medium can make such dosage forms. Absorption enhancers can also be used to increase the flux of the compound across the skin. Providing either a rate controlling membrane or dispersing the compound in a polymer matrix or gel can control the rate of such flux.

Examples of suitable aqueous and non-aqueous carriers, which may be employed in the pharmaceutical compositions of the disclosure, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/or antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In certain embodiments, a described compound or pharmaceutical preparation is administered orally. In other embodiments, a described compound or pharmaceutical preparation is administered intravenously. Alternative routes of administration include sublingual, intramuscular, and transdermal administrations.

When compounds described herein are administered as pharmaceuticals to humans and animals, they can be given per se or as a pharmaceutical composition containing, for example, 0.1% to 99.5% (more preferably, 0.5% to 90%) of active ingredient in combination with a pharmaceutically acceptable carrier.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc.; administration by injection, infusion or inhalation; topical by lotion or ointment; and rectal by suppositories.

Such compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, compounds described herein which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of the disclosure may be varied so as to obtain an amount of the active ingredient that is effective to achieve the desired therapeutic response for a particular patient, composition, and mode of administration, without being toxic to the patient.

The compositions described herein may contain from 0.1% to 99% by weight, preferably from 10 to 60% by weight, of the active material, depending on the method of administration.

Compositions may, if desired, be presented in a pack or dispenser device which may contain one or more unit dosage forms containing the active ingredients. The pack may, for example, comprise metal or plastic foil, such as a blister pack. Where the compounds are intended for administration as two separate compositions, these may be presented, for example, in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination by means of a single patient pack, or patient packs of each composition, including a package insert directing the patient to the correct use of the combination is a desirable additional embodiment.

According to a further embodiment, there is provided a patient pack comprising at least one active ingredient of the combination and an information insert containing directions on the use of the combination.

Embodiments of the disclosure are not limited to any particular agent encompassed by the classes of agents described above, and any agent that falls within any of these categories may be utilized in embodiments of the disclosure. Non-limiting examples of such agents are provided for clarity. Any of the secondary agents described above may be useful in embodiments of the disclosure.

The embodiments for disease state, subject type, daily dose amounts, therapeutically effective amounts, no observable adverse effect level dose amounts, non-effective dose amounts, pharmaceutical compositions, and chiral purities for the methods of the disclosure, which are described herein separately for the sake of brevity, can be joined in any suitable combination.

### Dosages

The compounds described herein may be administered to a patient at therapeutically effective doses to prevent, treat, or control one or more diseases described herein, such as but not limited to, the neurological diseases described herein. Pharmaceutical compositions comprising one or more of compounds described herein may be administered to a patient in an amount sufficient to elicit an effective therapeutic response in the patient. An amount adequate to accomplish this is defined as “therapeutically effective dose.” The dose can be determined by the efficacy of the particular compound employed and the condition of the subject, as well as the body weight or surface area of the area to be treated. The size of the dose also will be determined by the existence, nature, and extent of any adverse effects that accompany the administration of a particular compound or vector in a particular subject.

Toxicity and therapeutic efficacy of such compounds can be determined by standard pharmaceutical procedures in cell cultures or experimental animals, for example, by determining the LD50 (the dose lethal to 50% of the population) and the ED50 (the dose therapeutically effective in 50% of the population). The LD50 and the ED50 can be determined for the components alone or the combination. The dose ratio between toxic and therapeutic effects is the therapeutic index and can be expressed as the ratio, LD50/ED50. In some embodiments, combinations that exhibit large therapeutic indices are used. While compounds that exhibit toxic side effects can be used, care should be taken to design a delivery system that targets such compounds to the site of affected tissue to minimize potential damage to normal cells and, thereby, reduce side effects. The side effects can be avoided or reduced by using lower doses of one or more of the therapeutics.

The data obtained from cell culture assays and animal studies can be used to formulate a dosage range for use in humans. In some embodiments, the dosage of such compounds lies within a range of circulating concentrations that include the ED50 with little or no toxicity. The dosage can vary within this range depending upon the dosage form employed and the route of administration. For any compound described herein, the therapeutically effective dose can be estimated initially from cell culture assays. A dose can be formulated in animal models to achieve a circulating plasma concentration range that includes the IC50 (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a modulator is from about 1 ng/kg to 10 mg/kg for a typical subject.

The amount and frequency of administration of the compounds described herein and/or the pharmaceutically acceptable salts thereof will be regulated according to the judgment of the attending clinician considering such factors as age, condition and size of the patient as well as severity of the symptoms being treated. An ordinarily skilled physician or veterinarian can readily determine and prescribe the effective amount of the drug required to prevent, counter or arrest the progress of the condition. In general it is contemplated that an effective amount would be from 0.001 mg/kg to 10 mg/kg body weight, and in particular from 0.01 mg/kg to 1 mg/kg body weight. It may be appropriate to administer the required dose as two, three, four or more sub-doses at appropriate intervals throughout the day. Said sub-doses may be formulated as unit dosage forms, for example, containing 0.01 to 500 mg, and in particular 0.1 mg to 200 mg of active ingredient per unit dosage form.

In some embodiments, the pharmaceutical preparation is in a unit dosage form. In such form, the preparation is subdivided into suitably sized unit doses containing appropriate quantities of the active component, e.g., an effective amount to achieve the desired purpose. The quantity of active compound in a unit dose of preparation may be varied or adjusted from about 0.01 mg to about 1000 mg, from about 0.01 mg to about 750 mg, from about 0.01 mg to about 500 mg, or from about 0.01 mg to about 250 mg, according to the particular application. The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

In some embodiments, the therapeutically effective amount of the 5-HT6 receptor antagonist is from about 0.001 mg to about 1,000 mg. In some embodiments, the therapeutically effective amount of the 5-HT₆ receptor antagonist is from about 0.001 mg to about 175 mg. In some embodiments, the therapeutically effective amount of the 5-HT₆ receptor antagonist is from about 0.001 mg to about 70 mg. In some embodiments, the 5-HT₆ receptor antagonist is intepirdine or pharmaceutically acceptable salts, hydrates or solvates thereof. In some embodiments, intepirdine or pharmaceutically acceptable salts, hydrates or solvates thereof, is administered in a therapeutically effective amount. In some embodiments, a therapeutically effective amount of intepirdine, or pharmaceutically acceptable salts, hydrates or solvates thereof, is an amount of about 0.001 mg to about 1,000 mg. In some embodiments, a therapeutically effective amount of intepirdine, or pharmaceutically acceptable salts, hydrates or solvates thereof, is an amount of about 0.001 mg to about 500 mg. In some embodiments, a therapeutically effective amount of intepirdine, or pharmaceutically acceptable salts, hydrates or solvates thereof, is an amount of about 0.001 mg to about 200 mg. In some embodiments, a therapeutically effective amount of intepirdine, or pharmaceutically acceptable salts, hydrates or solvates thereof, is an amount of about 0.001 mg to about 35 mg. In some embodiments, a therapeutically effective amount of intepirdine, or pharmaceutically acceptable salts, hydrates or solvates thereof, is an amount of about 0.001 mg to about 175 mg. In some embodiments, a therapeutically effective amount of intepirdine, or pharmaceutically acceptable salts, hydrates or solvates thereof, is an amount of about 5 mg. In some embodiments, a therapeutically effective amount of intepirdine, or pharmaceutically acceptable salts, hydrates or solvates thereof, is an amount of about 15 mg. In some embodiments, a therapeutically effective amount of intepirdine or pharmaceutically acceptable salts, hydrates or solvates thereof is an amount of about 35 mg. In some embodiments, a therapeutically effective amount of intepirdine or pharmaceutically acceptable salts, hydrates or solvates thereof is an amount of about 70 mg. In some embodiments, the 5-HT₆ receptor antagonist is administered orally. In some embodiments, intepirdine or pharmaceutically acceptable salts, hydrates or solvates thereof, is administered orally. In some embodiments, the 5-HT₆ receptor antagonist is administered daily. In some embodiments, intepirdine or pharmaceutically acceptable salts, hydrates or solvates thereof, is administered daily.

In some embodiments, one or more compounds described herein are administered with another compound. The administration may be sequentially or concurrently. The combination may be in the same dosage form or administered as separate doses. In some embodiments, the first compound is a 5-HT₆ receptor antagonist and the other compound is one or cholinesterase inhibitors. In some embodiments, the cholinesterase inhibitor is administered before the 5-HT₆ receptor antagonist. In some embodiments, the subject is on stable cholinesterase inhibitor therapy. In some embodiments, the subject has been on a stable background cholinesterase inhibitor therapy for at least 60 days prior to treatment and may have received a cholinesterase inhibitor at any dose for at least four months prior to prior to administering intepirdine. In some embodiments the acetylcholinesterase inhibitor is donepezil, or pharmaceutically acceptable salts, hydrates or solvates thereof. In some embodiments, donepezil, or pharmaceutically acceptable salts, hydrates or solvates thereof is administered to a subject in need thereof, in a therapeutically effective amount. In some embodiments, donepezil, or pharmaceutically acceptable salts, hydrates or solvates thereof, is administered to a subject in need thereof in a daily dose of about 1 mg to about 25 mg. In some embodiments, donepezil, or pharmaceutically acceptable salts, hydrates or solvates thereof, is administered to a subject in need thereof in a daily dose of about 5 mg, 10 mg or 23 mg. In some embodiments, donepezil, or pharmaceutically acceptable salts, hydrates or solvates thereof, is administered to a subject in need thereof in a daily dose that is considered to be subtherapeutic. In some embodiments, donepezil, or pharmaceutically acceptable salts, hydrates or solvates thereof, is administered to a subject in need thereof in an amount of about 1 mg to about 25 mg. In some embodiments, donepezil, or pharmaceutically acceptable salts, hydrates or solvates thereof, is administered to a subject in an amount of about 5 mg, 10 mg or 23 mg. In some embodiments, donepezil, or pharmaceutically acceptable salts, hydrates or solvates thereof, is administered to a subject in need thereof in an amount that is considered to be subtherapeutic. A “subtherapeutic amount” refers to a dosage that is below that typically used for the subject agent in typical therapeutic or prophylactic use.

### EXAMPLES

The following examples are illustrative, but not limiting, of the methods and compositions described herein. Other suitable modifications and adaptations of the variety of conditions and parameters normally encountered in therapy and that are obvious to those skilled in the art are within the spirit and scope of the compounds and methods described herein.

### Example 1

### A Phase 2, Double-Blind, Randomized, Placebo-Controlled Crossover Study Evaluating the Effect of Intepirdine on Gait and Balance in Patients with Dementia and Experiencing Falls

Primary Objective: To assess the effect of intepirdine versus placebo on gait speed, a quantitative measure of functional mobility, evaluated under normal and dual-task walking conditions on an electronic walkway system after 2 weeks of treatment.

Secondary Objection: To assess the safety and tolerability of intepirdine.

Exploratory Objectives: To assess the effect of intepirdine versus placebo on gait variability, evaluated under normal and dual-task walking conditions on an electronic walkway system after 2 weeks of treatment; to assess the effect of intepirdine versus placebo on gait and balance, as measured by the mini-BESTest battery, after 2 weeks of treatment; to assess the effect of intepirdine versus placebo on gait and balance, as measured by individual item scores calculated by Opal APDM sensors during performance of the mini-BESTest battery, after 2 weeks of treatment; to assess the effect of intepirdine versus placebo on freezing of gait (FOG), after 2 weeks of treatment; to assess the effect of intepirdine versus placebo on movement and balance, as measured by the Motor Disorder Society Unified Parkinson's Disease Rating Scale (MDS-UPDRS) Part III and PIGD subscore, after 2 weeks of treatment; and to assess the effect of intepirdine versus placebo on smell, a proxy for cholinergic function, as measured by the University of Pennsylvania Smell Identification Test (UPSIT), after 2 weeks of treatment.

Approximately 30-40 patients with dementia (AD, DLB, or PDD) and gait impairment will be enrolled. Subjects must be on a stable regimen of a cholinesterase inhibitor for at least two months prior to screening, and must have received a cholinesterase inhibitor for at least four months. Subjects may be on certain other background therapies if on a stable regimen prior to screening. These allowed background therapies and stability requirements are listed in Table 1.

Key Inclusion Criteria: Subjects eligible for enrollment in the study must meet all of the following criteria: Male or female subject with a clinical diagnosis of Alzheimer's disease (AD), dementia with Lewy bodies (DLB), or Parkinson's disease dementia (PDD). The diagnosis of AD must be in accordance with the recommendations from the National Institute on Aging-Alzheimer's Association workgroups on diagnostic guidelines for AD (McKhann et al., 2011). The diagnosis of PDD must be in accordance with the Movement Disorder Society (MDS) criteria for PDD (Emre et al., 2007). The diagnosis of DLB must be in accordance with Consensus criteria for DLB diagnosis (McKeith et al., 2005). Subject has an MMSE score 14 to 26 inclusive, at both the Screening and Baseline Visits. Subject must have experienced at least one fall within the past two years as assessed by history and defined by the judgement of the clinical investigator at the time of the Screening Visit. Clinically relevant risk of fall. For example, patients have an average gait speed of less than 100 cm/s on an electronic walkway evaluation or abnormal gait and balance on the miniBEST assessment with a score less than 16. In case these criteria are not met, a justification for the clinically determined fall risk should be documented and discussed with the medical monitor. Other documented risks of fall or gait impairment may be acceptable with approval from the medical monitor. Mandatory need for a walking aid could for example be considered as such a condition. Subject has a Hachinski Ischaemia score less than or equal to 4 at Screening. Subject has a documented history of at least 6 weeks of stable cholinesterase inhibitor therapy prior to screening with no intent to change for the duration of the study. If the subject is receiving dopaminergic therapy for parkinsonism or other motor impairment, the subject must have a documented history of at least 60 days of therapy at a stable dose prior to the Screening Visit and there is no intent to change the dose for the duration of the study. Age 50 to 89 years, inclusive, at the time of the Screening Visit. Female subjects must be: Of non-childbearing potential (i.e., any female who is post-menopausal [greater than 1 year without menstrual period in the absence of hormone replacement therapy]) or surgically sterile; or, If pre-menopausal or menopausal for 1 year or less, must have a negative pregnancy test and must not be lactating at the Screening and Baseline Visits. Female subjects of childbearing potential and who are sexually active are required to practice adequate methods of birth control. Female subjects for whom menopausal status is in doubt in the opinion of the investigator will be required to use an adequate form of birth control. Acceptable, adequate form of birth control is defined as consistent use of combined effective methods of contraception including at least one barrier method. Male subjects who are sexually active are also be required to use an adequate form of birth control as described above. Subject has the ability to comply with procedures for evaluating gait, balance, and other testing in the opinion of the investigator. Subject must be able to ingest pills (in tablet form) whole; Subject lives with (or has substantial periods of contact with) a regular caregiver or healthcare provider who is willing to attend visits, oversee the subject's compliance with protocol-specified procedures and study medication, and report on subject's status, and who has substantial contact with the subject. If the caregiver does not cohabitate with the subject, he/she ideally should have a minimum of 10 hours total and at least 3 days contact with the subject per week. Prior to randomization, study representatives will review eligibility of non-cohabitating caregivers. Every effort should be made to have the same caregiver/healthcare provider throughout the study; Subject has provided full written informed consent prior to the performance of any protocol specified procedure; or if unable to provide informed consent due to cognitive status, subject has provided assent and a legally acceptable representative has provided full written informed consent on behalf of the subject; Caregiver has provided full written informed consent on his/her own behalf prior to the performance of any protocol-specified procedure; General health status is acceptable for participation for the entire duration of the study.

Key Exclusion Criteria: A subject will not be eligible for inclusion in this study if any of the following criteria apply: Other Causes for Dementia: History and/or evidence of any other CNS disorder that could be interpreted as a cause of dementia (in the opinion of the investigator), e.g., cerebrovascular disease (transient ischemic attack, stroke, hemorrhage); structural or developmental abnormality; epilepsy; infectious, degenerative, or inflammatory/demyelinating CNS conditions; Evidence of the following disorders where this is thought to be the cause of, or to contribute to the severity of, the subject's dementia: current vitamin B12 deficiency, hypothyroidism, neurosyphilis, or Wernicke's encephalopathy. Atypical clinical features or clinical course of dementia that would lead the investigator to conclude symptoms are more likely due to an alternate dementia diagnosis other than AD, DLB, or PDD. Confounding Medical Conditions: History of significant psychiatric illness such as schizophrenia or bipolar affective disorder or any other significant psychiatric illness that in the opinion of the investigator would interfere with participation in the study; history of major depressive disorder in the past year, or current major depressive episode. Significant suicide risk as defined by (1) suicidal ideation as endorsed on items 4 or 5 on the C-SSRS within the past year, at Screening or at Baseline; (2) suicidal behaviors within the past year; (3) clinical assessment of significant suicidal risk during subject interview. Current psychosis that in the opinion of the investigator would interfere with the subject's ability to participate in this study. History of epilepsy or unexplained seizure in the past 5 years, unexplained recent loss of consciousness, or history of significant head trauma with loss of consciousness. History of malignancy during the 5 years before Screening. History of basal cell carcinoma and melanoma in situ are permitted. History of other cancers currently in a non-active state may be acceptable after review with the Medical Monitor. Any clinically relevant concomitant disease including progressive liver or kidney dysfunction, history of myocardial infarction or unstable angina within 6 months of Screening, history of more than 1 myocardial infarction within 5 years of Screening, history of clinically significant stroke, or any other medical or psychiatric condition, which, in the opinion of the investigator, makes the subject unsuitable for inclusion in the study. History of alcohol use disorder or other substance abuse disorder (excluding tobacco use), according to the Diagnostic and Statistical Manual of Mental Disorders, 5th Edition, criteria in the past 10 years. History of Down syndrome or mental retardation. Severe and/or debilitating arthritis or peripheral neuropathy. Peripheral neuropathy should be assessed quantitatively with a 128-Hz clanging tuning fork test applied to the hallux of both feet. Vibration sensation by the subject for 4 seconds or less will be excluded due to severe neuropathy. Significant tremor which would interfere with recording balance and walking. Uncorrected vision disturbance or vestibular problems that would affect gait and balance. Symptomatic bradycardia which in the opinion of the investigator may place the subject at risk of syncope. Concomitant Medications: Participation in another investigational drug or device study in dementia during the 60 days prior to the Screening Visit (Visit 1), or within 5 half-lives of use of the investigational drug prior to the Screening Visit, whichever is longer. In addition, subjects who were previously screened for another study in dementia but failed the entry criteria for that study may be screened with no time delay prior to the Screening Visit, provided that, in the opinion of the investigator, and after review with the Medical Monitor, there is a realistic possibility that the subject would be eligible.

Permitted Medications and Non-Drug Therapies: All concomitant medications and those taken within 6 months prior to Screening, including over-the-counter and herbal remedies, will be recorded in the eCRF. Non-medication therapies related to the subject's dementia (e.g. neurostimulation, cognitive rehabilitation) that have occurred in the 12 months prior to Screening must also be recorded. The name of the drug, the dose, indication and route of administration as well as the dates administered should be documented; the minimum requirement is to record the drug name and dates of administration. Any medication not specified in the list of prohibited and conditional medications provided in Table 1 is permitted during the study. Treatment with any concomitant medications as detailed in Table 1, unless otherwise specified, need to have been discontinued for 5 half-lives prior to screening and assessed as no longer clinically necessary for the subject.

TABLE 1
List of Prohibited and Conditional Concomitant Medications

| | Conditional Medications: |
|---|---|
| Prohibited Medications: | Not allowed unless prescribed at a stable dose |
| Not allowed during the study or within 5 half- | for at least 2 months prior to Screening; dose |
| lives prior to Screening | during the study should be stable, if possible |
| Butyrophenones, phenothiazines, and other | Acetylcholinesterase inhibitors |
| “conventional” antipsychotics | Selective MAO-B inhibitors (rasagiline, |
| Barbiturates | selegiline) |
| Non selective MAO inhibitors | Memantine |
| Substrates of CYP2C9¹ with narrow | Axona ® (caprylidene) |
| therapeutic indices: warfarin, phenytoin and | Antidepressants (other than MAO inhibitors) |
| (R)-acenocoumarol (active component of | Thyroid hormones |
| some non-warfarin anticoagulants) | Atypical antipsychotics (e.g., risperidone) |
| Potent CYP3A4² inhibitors/inducers such as | Pimavanserin |
| ketoconazole, itraconazole, erythromycin, | Stable mediciation with benzodiazepines and |
| rifampicin, phenytoin and carbamazepine | other sedatives/hypnotics |
| Known potent Pgp inhibitors³ (itraconazole, | Note: Benzodiazepines or other |
| ketoconazole, cyclosporin, diltiazem, | sedatives/hypnotics (including |
| verapamil, quinidine, and carvedilol) | antihistamines) with half-life less than |
| Any investigational drug | 6 hours can be taken on an as-needed basis |
| Trans-Magnetic Stiumulation (TMS) | but must not be taken within 5 half-lives |
| Marijuana/THC | prior to cognitive testing. |
| | Dopaminergic therapies for motor |
| | impairment (other than MAO inhibitors) |
| | Deep Brain Stimulation (DBS); Note: DNS |
| | settings, parameters, and regimen must be |
| | stable for at least 60 days prior to screen visit |
| | Cognitive or motor tasks for cognitive or |
| | motor rehabilitation performed under medical |
| | supervision, including occupational |
| | rehabilitation and/or physical therapy that |
| | may impact gait and balance |
| | Thrombocyte aggregation inhibitors (low |
| | dose ASS, clopidogrel) |

Unacceptable Test/Laboratory Values: Persistent or recurrent liver enzyme elevations, alanine transaminase (ALT) and/or aspartate aminotransferase (AST) greater than or equal to 2.0 times upper limit of normal (ULN). Total bilirubin over 1.5×ULN except due to documented Gilbert's disease. Calculated creatinine clearance less than 40 mL/min (Cockroft-Gault formula): Adult males: [(140—age in years)×(weight in kg)]±72×serum creatinine* Adult females: 0.85 x [((140—age in years)×(weight in kg))±72×serum creatinine*]*in mg/dL. Positive hepatitis B surface antigen or hepatitis C antibody test. Confirmed corrected QT interval (QTc) value greater than or equal to 450 msec for males or greater than or equal to 470 msec for females. Subjects with a QRS value greater than 120 msec and QTc value less than 500 msec may be eligible following discussion with the Medical Monitor. Clinically relevant renal dysfunction: plasma creatinine >1.5 mg/dl, when considered clinically significant.

Other ineligibility criteria: Previous exposure to intepirdine or SB742457. Subject is unable to take study medication as prescribed throughout the study (with assistance is acceptable), has a significant history of non-compliance with prescribed medication, or is at risk of non-compliance with study medication or procedures. Subject or caregiver is an immediate family member or employee of the participating investigator, any of the participating site staff, or of the sponsor study staff. Subject was prescribed cognitive tasks for cognitive rehabilitation performed under medical supervision in the 3 months prior to Screening and/or during the study. Subject has participated in a program of neurostimulation in the past 3 months or plans to participate in a program of neurostimulation during the course of the study.

Investigational Product (IP) and Other Study Treatment: IP in this study is defined as 35 mg intepirdine tablets and their matching placebo, and will be provided by Axovant Sciences. intepirdine and placebo tablets will be indistinguishable from each other. All subjects will take one tablet of IP daily at bedtime, with or without food; this will consist of either one tablet of 35 mg intepirdine or one tablet of placebo. See Table 2.

IP for the Single-Blind Run-In Period (Weeks −2 to 0) will be supplied at Visit 2. Subjects will be given an IP kit containing 1 bottle of placebo tablets. Each bottle will contain 50 tablets, which is sufficient medication for 2 weeks plus 36 days overage. Subjects will be instructed to take one tablet of IP daily at bedtime with or without food. In the event that the Single-Blind Run-In period is extended, no new IP will be dispensed. Drug accountability will be checked, and subjects will be re-dispensed the study medication kit and bottles they received at Visit 2.

IP for the Double-Blind Treatment Period (Weeks 0 to 8) will be supplied at Visits 3, 4, and 5. At each dispensing visit during the Treatment Period, subjects will be given an IP kit containing one bottle of IP. Depending on treatment assignment, subjects will receive either one bottle of 35 mg intepirdine tablets or one bottle of placebo tablets. Each bottle will contain 50 tablets, which is sufficient for 4 weeks plus 22 days overage. Subjects will be instructed to take one tablet of IP daily at bedtime with or without food. New bottles of IP will not be dispensed at Visit 6, however, drug accountability will be checked. All subjects and their caregivers should be instructed to bring IP bottles, with any unused drug, to each visit.

TABLE 2
Investigational Product and Other Study Treatment

| | | |
|---|---|---|
| Product name: | Investigational | Placebo |
| | Product | |
| | intepirdine | |
| Formulation description: | pink, film-coated, | pink, film-coated, |
| | round tablets | round tablets |
| Dosage form: | 35 mg Tablet | Placebo Tablet |
| Unit dose strength(s)/ | 35 mg | N/A |
| Dosage level(s): | 35 mg | placebo |
| Route of Administration: | Oral | Oral |
| Duration (Run-in Period): | N/A | 2 weeks |
| Duration (Treatment Period): | 2 weeks | 2 weeks |
| Dosing instructions: | Take before | Take before |
| | bedtime with or | bedtime with or |
| | without food | without food |
| Manufacturer/Source of | Metrics Contract | Metrics Contract |
| Procurement | Services | Services |
| | Greenville, NC | Greenville, NC |

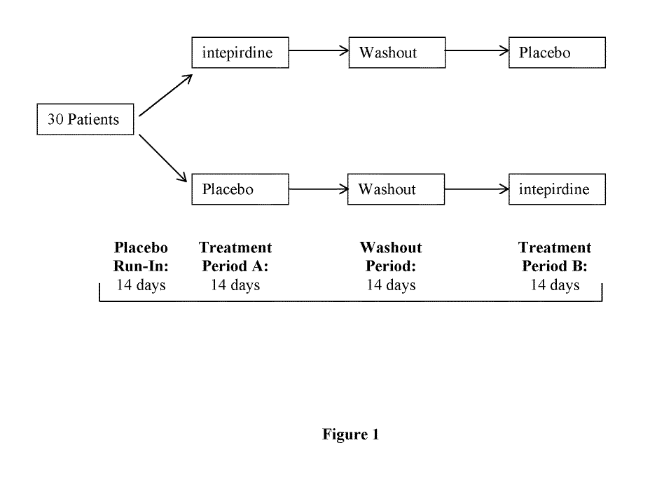

Randomization/Treatment Assignment: FIG. 1 graphically depicts the study design. Subjects will be assigned to receive 35 mg RVT 101 or placebo in accordance with the randomization schedule, prior to the start of the Double-Blind Treatment Period, using validated software. Following confirmation of eligibility at Visit 3 (end of Single-Blind Run-In Period), subjects will be randomized to placebo or 35 mg RVT 101 in a 1:1 ratio. Each site will have an approximately 1:1 ratio of patients randomized to intepirdine and placebo.

Blinding: This will be a double-blind study. The study will include a 2-week Single-Blind Run-In Period during which investigators will know that the subject is taking placebo but the subject/caregiver will not. This will be followed by a 6-week Double-Blind Treatment Period consisting of three parts: a 2-week Treatment Period B, a 2-week Washout Period, and a 2-week Crossover Treatment Period C. During the Double-Blind Treatment Period, when neither subjects nor investigators will know which of the two treatments the subject is receiving. Subjects and caregivers will not be informed of transition from the Single-Blind Run-In Period to the Double-Blind Treatment Period. RVT 101 and placebo will be provided as tablets that are indistinguishable in appearance, smell, and taste.

The following will apply: The investigator or treating physician may unblind a subject's treatment assignment. Such a measure should be taken only in the case of an emergency OR in the event of a serious medical condition when knowledge of the investigational product is essential for the appropriate clinical management or welfare of the subject as judged by the investigator. In the event that a medical emergency or condition requires knowledge of the subject's treatment assignment, the investigator will contact the medical monitor to discuss the potential need for unblinding. The procedure of unblinding for a specific subject is provided in the Study Reference Manual. If, for safety reasons, the investigator determines that the subject's treatment must be immediately unblinded to provide adequate medical treatment, the investigator must inform the Medical Monitor about the unblinding as soon as possible, but without revealing the treatment assignment of the unblinded subject. The sponsor will be informed without delay of the decision to unblind any subject and will determine whether any additional measures need to be taken for the safety of subjects currently in the study. Any other requests to reveal a subject's treatment identity must be requested of, and approved by, Axovant Sciences. A subject will be withdrawn from the study if his or her treatment code is unblinded by the investigator or treating physician. The date and reason for the unblinding must be fully documented in the eCRF. Axovant Sciences or their designee may unblind the treatment assignment for any subject if this is required to fulfill regulatory reporting obligations such as expedited SAE reporting.

Time and Events: The Time and Events Schedule (Table 3) displays each study assessment and procedure along with the time of occurrence. All study assessments should be conducted by the investigator, and/or a suitably qualified designee approved and documented for this study. All raters will be trained and certified to perform the specific rating scales in this study.

Visit 3 will occur within 14 to 17 days after Visit 2. For Visits 4 through 6, there is a visit window of +3 days. If the visit window is used, the subsequent visit should be scheduled according to the date when the prior study visit actually took place and not according to the original visit schedule (i.e., the subsequent visit date should be re-calculated from the date of the previous visit). Information will be recorded in the source documents and, where appropriate, the case report form. If, during the visit, the subject is unable or, in the judgment of the investigator, unlikely to be able to complete the cognitive assessments, the testing may be rescheduled within the windows described previously. Subjects may be given breaks during the assessments or may have assessments split across different days so long as all assessments are completed within the visit window. Individual assessments should, however, be completed within a single day. Assessment should occur in the following order whenever possible: MMSE (screening only), Modified Hachinski Ischemia Scale (mHIS, screening only), motor testing (electronic walkway tests, miniBESTest, FOG assessment, for patients with Lew Body dementia who are in the “off” state or show motor fluctuations, motor testing should be done during the “on” phase), caregiver FES (Screening only, UPSIT, C-SSRS, MDS-UPDRS, physical and neurological exam, 12-lead ECG, vital signs, blood draws.

TABLE 3
Time and Events Schedule

| | Visit Description | | | | | | | |
|---|---|---|---|---|---|---|---|---|
| | | Beginning | Beginning | Beginning | Beginning | End of | Follow-up | |
| | Screening | of Period 1 | of Period 2 | of Period 3 | of Period 4 | Study | Visit | ET |
| Study Visit Number | V1 | V2 | V3¹ | V4 | V5 | V6 | V7 | |
| Study Week | W (−6) | W (−2) | W 0 | W 2 | W 4 | W 6 | W 8 | |
| Study Day | Up to 28 + | 14 + 3 days | 0 | 14 ± 3 | 28 ± 3 | 42 ± 3 | 58 + 5 | |
| | 14 days | before V3 | | | | | | |
| | before V2 | | | | | | | |
| Informed consent | X | | | | | | | |
| Inclusion/Exclusion criteria | X | X | X | | | | | |
| Demography | X | | | | | | | |
| Medical History | X | | | | | | | |
| Concomitant medications | X | X | X | X | X | X | | X |
| Blood alcohol and urine drug | X | | X | | | | | |
| screen | | | | | | | | |
| C-SSRS, Screening version | X | | | | | | | |
| C-SSRS, Since Last Visit | | X | X | X | X | X | | X |
| version | | | | | | | | |
| Randomization | | | X | | | | | |
| Dispense IP | | X | X | X | X | | | |
| Assess IP compliance | | | X | X | X | X | | X |
| Physical exam | X | X | X | X | X | X | | X |
| Complete neurological exam | X | X | X | X | X | X | | X |
| MRI or CT | X | | | | | | | |
| 12-Lead ECG | X | X | X | X | X | X | | X |
| Vital signs² | X | X | X | X | X | X | | X |
| Review Adverse Events | X | X | X | X | X | X | X | X |
| Serum OR urine pregnancy | X | | X | | | | | X |
| test³ | | | | | | | | |
| Urine pregnancy test | | | | | | X | | |
| Hep B and Hep C screen⁴ | X | | | | | | | |
| TSH, vitamin B₁₂, syphilis | X | | | | | | | |
| serology⁵ | | | | | | | | |
| Serum chemistry | X | X | X | X | X | X | | X |
| Hematology | X | X | X | X | X | X | | X |
| Urinalysis | X | X | X | X | X | X | | X |
| MMSE | X | | | | | | | |
| UPSIT | X | | X | X | X | X | | |
| Primary Gait Screen: | X | X | X | | | | | |
| assessment of gait speed | | | | | | | | |
| Single and dual task gait | X | X | X | X | X | X | | |
| assessments | | | | | | | | |
| Mini BESTest assessment | X | X | X | X | X | X | | |
| Freezing of gait evaluation | | X | X | X | X | X | | |
| MDS-UPDRS Parts II and III | | X | X | X | X | X | | |
| Caregiver-adapted Falls | X | | | | | | | |
| Efficacy Scale | | | | | | | | |
| Handgrip strength evaluation | X | | | | | | | |
| Hachinski Ischemia Scale | X | | | | | | | |

Abbreviations: ECG = electrocardiogram; Hep = hepatitis; IP = investigational product; mini-BESTest = mini Balance Evaluation Systems Test; MMSE = mini-mental state examination; TSH = thyroid stimulating hormone; MDS-UPDRS = Movement Disorder Society - Unified Parkinson's Disease Rating Scale; UPSIT = University of Pennsylvania Smell Identification Test
¹Early Termination visit should be performed within 7 days of stopping study drug if permanently discontinued between scheduled clinic visits.
²If the visit window is used, the subsequent visit should be scheduled according to the date when the prior study visit actually took place and not according to the original visit schedule (i.e., the subsequent visit date should be re-calculated from the date of the previous visit).
³Complete physical exam will be performed at Screening, V6, and ET visit and include, at a minimum, assessment of the cardiovascular, respiratory, gastrointestinal, and neurological systems. In addition, the following body systems should be assessed, if possible: skin, head, eyes, ears, nose, neck/throat, endocrine/metabolic, genitourinary, blood/lymphatic, musculoskeletal, and abdomen (liver and spleen).
⁴Brief, symptoms-directed physical examination will be performed at V2, V3, V4, and V5 and include, at a minimum, assessments of the head, eyes, ears, nose, neck/throat, lungs, cardiovascular system, and abdomen (liver and spleen) lungs, cardiovascular system, and abdomen (liver and spleen).
⁵Neurological examinations will include assessment of gait, balance, coordination, cranial nerves and motor and sensory systems, including assessment of peripheral neuropathy.
6. MRI or CT will be performed between V1 and V2 if no CT or MRI scan has been performed within the previous five years since diagnosis. These scan findings must be consistent with the diagnosis of AD, DLB, or PDD without any other clinically significant pathologies.
7. Vital signs will include systolic blood pressure, diastolic blood pressure, heart rate, respiratory rate, and temperature and body weight at each visit and height at screening. Postural changes in blood pressure and heart rate will also be assessed at every visit.
8. Required only for women of child bearing potential. Either a serum or urine pregnancy test is to be performed at V1, V3, and Early Termination. A urine pregnancy test is to be performed at Visit 6. If a urine pregnancy test result is positive, an unscheduled serum pregnancy test must be performed to confirm the results.
9. If these tests were performed within 3 months prior to the planned first dose of investigational product, testing is not required. Records must be present in the subject's source documents.
10. If these tests were performed within 12 months prior to the planned first dose of investigational product, testing is not required. Records must be present in the subject's source documents.
11. Note:
for patients with Lewy body dementia (dementia with Lewy bodies or Parkinson's disease dementia) who show motor fluctuations, motor testing should be done during the “on” phase.
12. Patients who require assistive devices and meet the criteria for clinically relevant risk of falls may not perform the PGS.

Screening Period (up to 28 days before Visit 2): Subjects will be screened for eligibility during the Screening Period. Subjects who do not qualify for the study during this period will be considered screen failures. An ICF will be signed by each subject, if they are able, or by the caregiver with subject assent. An ICF will also be signed by the caregiver before any study-specific procedures are performed. Subjects will be screened according to study inclusion/exclusion criteria. This Screening Period may be extended for up to an additional 14 days if needed to complete assessment activities after approval by the study Medical Monitor. Subjects who are screen failures during the Screening Period may be rescreened after discussion with the Medical Monitor. Note: Subjects who are screen failures may be rescreened only once.

At the screening visit, all potential subjects will undergo MMSE assessment and gait speed evaluation on an electronic walkway system. If possible, the MMSE should be the first assessment performed, followed by the electronic walkway assessment. If the MMSE score or gait speed do not meet the criterion for randomization at screening, no other assessments should be performed.

Single-Blind Run-In Period (14+5 days before Visit 3): At Visit 2, subjects who meet all study screening criteria will enter a Single-Blind Run-In Period. Investigational product will be dispensed. Subjects will be instructed to take the investigational product once daily in the morning. Subjects will be instructed to take the first Single-Blind Run-In investigational product (single-blind placebo) during the study visit. Visit 2 assessments will be performed according to Table 3. To qualify for randomization at Baseline (Visit 3) subjects must return unused study medication, be considered capable of completing study assessments, remain within study-specified criteria for MMSE, meet criteria for having slow gait as described in the protocol (i.e. gait speed below 100 cm/s on electronic walkway analysis), and meet all other eligibility requirements.

The Single-Blind Run-In Period may be extended for up to 3 weeks after discussion with the Medical Monitor for subjects who do not meet the gait speed threshold necessary for randomization at Visit 3, and may be re-evaluated one additional time during this extension to determine eligibility. This subsequent visit will then serve as the subject's baseline if randomization criteria are met. No new investigational product will be dispensed for the additional run-in period; subjects should be instructed to continue taking blinded investigational product dispensed at Visit 2. The MMSE should be the first assessment performed, followed by the electronic walkway assessment, at the baseline visit. If the MMSE score or gait speed do not meet the criterion for randomization at Visit 3, no other assessments should be performed. If after discussion with the Medical Monitor it is determined that the Single-Blind Run-In Period may be extended by 3 weeks per the criterion above, the subject should be scheduled to return to the clinic in 3 weeks' time (+3 days), and all baseline assessments should be performed at this repeat visit.

Baseline (Visit 3) and Double-blind Treatment (Period B) (Visit 3-Visit 4): At Visit 3 (Baseline), assessments will be performed to determine subject eligibility. Eligible subjects will be randomized 1:1 to one of the following treatment sequences: AP=intepirdine in Period B and placebo in Period C; PA=placebo in Period B and intepirdine in Period C. During this double-blind treatment period (Period B), investigational product will be dispensed at Visit 3. Subjects will ingest the first dose of investigational product in the clinic in the presence of study personnel. All additional doses will be ingested as outpatients. Subjects who prematurely discontinue double-blind investigational treatment in Period B should be encouraged to return to the clinic for an Early Termination Visit, and Visit 7 assessments and procedures will be completed.

Washout Period (Visit 4-Visit5): No investigational product will be given during the washout period. After the two-week washout, subjects will enter the second two-week treatment period (Period C). This period has a 5 day allowable window to accommodate scheduling conflicts. Subjects who prematurely discontinue from the washout period should be encouraged to return to the clinic for an Early Termination Visit, and Visit 7 assessments and procedures will be completed.

Double-blind Treatment (Period C) (Visit 5-Visit6): During the second two-week double-blind treatment period (Period C), subjects who received intepirdine during Period B will receive placebo; subjects who received placebo during Period B will receive intepirdine. During this double-blind treatment period (Period C), investigational product will be dispensed at Visit 5. Subjects will ingest the first dose of investigational product in the clinic in the presence of study personnel. All additional doses will be ingested as outpatients. Subjects who prematurely discontinue double-blind investigational treatment in Period C should be encouraged to return to the clinic for an Early Termination Visit, and Visit 7 assessments and procedures will be completed.

Critical Baseline Assessments: All baseline and efficacy assessments should be performed following the methodology included in the study manual. The procedures are briefly summarized below.

Mini-Mental State Evaluation (MMSE): The MMSE (Folstein et al., 1975) consists of 11 tests of orientation, memory (recent and immediate), concentration, language, and praxis. Scores range from 0 to 30, with lower scores indicating greater cognitive impairment. It is based on the performance of the subject and takes approximately 5 to 10 minutes to administer. The MMSE for patients included in the study must be between 14 and 26 inclusive at both the screening and baseline visits in order to qualify for randomization into the study.

Gait Speed Measured on Electronic Walkway Assessment: Electronic walkway systems, such as the GAITRite system, are computerized assessment tools that utilize an electronic walkway mat consisting of pressure-sensitive pads that can calculate spatiotemporal gait parameters including gait velocity, cadence, step time, step length, stride length, stride time, swing time, stance time, and double support time (Buesing et al., 2015). Subjects included in the study must have an average gait speed of less than 100 cm/s, or other clinically relevant risk of falls at Visits 1, 2 and 3. Subjects without assistive devices will undergo the Primary Gait Screen assessment (PGS) at Visits 1, 2 and 3, and all subjects will complete additional gait assessments under single and dual task conditions at all study visits. The specific procedures for the PGS and single and dual task gait assessments, including the calculation to determine study eligibility, are described in the Study Reference Manual.

University of Pennsylvania Small Identification Test (UPSIT): The olfactory system is a cholinergic rich area of the brain that degenerates early in PD. Approximately 90% of patients with early stage PD have olfactory dysfunction, and olfactory dysfunction correlates with loss of cholinergic neurons in structures such as the nucleus basalis of Meynert (nBM). The UPSIT is a 40 item ‘scratch-and sniff’ test that takes about 15 minutes to administer and is a reliable measure of olfactory function. The materials for the test will be provided to the sites, and the procedure for administering the test are described in the Study Reference Manual.

Caregiver-adapted version of the Fall Efficacy Scale: This scale will be rated by the caregiver and will capture his/her degree of concern about the likelihood of a patient falling while under a variety of different circumstances and performing different activities. The scale is adapted from the Falls Efficacy Scale—International, which is a 16-item self-reported scale in which individuals rate their degree of concern regarding the likelihood of their falling during different activities. The degree of concern is reflected in a 1-4 rating for each item, with “1” indicating that the individual is “not at all concerned” and “4” indicating that the individuals is “very concerned.” The scale has been reported to have strong internal validity (Cronbach's alpha=0.96) and test-retest reliability (ICC=0.96) (Yardley et al.,2005). A caregiver-adapted version of this scale will be used in this study and administered only at the screening visit.

Handgrip Strength Assessment: Low handgrip strength is a clinically relevant indicator of poor mobility ((Cruz-Jentoft et al., 2010). Moreover, baseline handgrip strength has been shown to have a linear relationship with risk of incident disability of activities of daily living (Al Snih et al., 2004). In this study, handgrip strength will be captured at screening. Handgrip strength will be assessed by a baseline hand dynamometer embedded with a strain gauge, as described on the Study Reference Manual.

Modified Hachinski Ischemia Scale (mHIS): This scale asks about the presence or absence of 8 clinical symptoms. It is being used in this study at Screening to identify and exclude subjects with vascular dementia.

Efficacy Assessments: All study assessments should be conducted by the investigator, and/or a suitably qualified designee, all of whom will be trained and certified to administer these measures for this study. Every effort should be made for the same person to conduct specific assessments on each individual subject at each study visit. Assessments may be monitored for quality. Screening assessments along with accompanying data will be reviewed to ensure that subjects meet the inclusion criteria. Other assessments will be monitored by using data collected.

Mini Balance Evaluation Systems Test (Mini-BESTest): The Mini-BESTest is a short, well-validated 14 item assessment of dynamic balance, specifically anticipatory postural transitions, postural responses, and dynamic gait (King et al., 2012). Each item is scored from (0-2) with a score of 2 being normal. The total score is out of 28. This test has excellent test-retest reliability (ICC=0.96) and interrater reliability (ICC=0.96) (Godi et al., 2013). Another advantage of the Mini-BESTest is its dual task walking assessment which is a reliable test of attention and executive function (Yarnall et al., 2011). The test takes about 10 minutes to administer (Mancini and Horak, 2010). The Mini-BESTest assessment will be performed with subjects wearing the Opal APDM sensors if available at the site, which will collect quantitative measures of dynamic gait and balance.

Electronic Walkway Assessment: Electronic walkway systems, such as the GAITRite system, are computerized assessment tools that utilize an electronic walkway mat consisting of pressure-sensitive pads that can calculate spatiotemporal gait parameters including gait velocity, cadence, step time, step length, stride length, stride time, swing time, stance time, and double support time (Buesing et al., 2015). Parameters such as gait velocity and cadence on the electronic walkway system are highly correlated with manually timed tests in PD patients (ICC=0.96, p<0.0005) (Bryant et al., 2013). Gait variability, specifically step length and step time, predict falls (Hausdorff, 2005) (Bryant et al., 2013). Stride length has been highly correlated with the PIGD-UPDRS subscore (see 6.3.1.3) (r=−0.90, p<0.0001) (Salarian et al., 2004). Further, electronic walkway systems are highly sensitive to demonstrating the effects of medications used to treat PD. The GAITRite system is able to detect gait changes with dopaminergic medications only 45 minutes after administration (Bryant et al., 2013). Recently, evidence has shown that cholinesterase inhibitors are also able to improve gait parameters in AD and PD (Henderson et al., 2016). Hence, the parameters measured by an electronic walkway system correlate well with established clinical scoring methods of gait, and these parameters are reliable and sensitive to capturing the effects of pharmacologic treatment. Subjects without assistive devices will undergo the Primary Gait Screen assessment (PGS) at Visits 1, 2 and 3, as well as additional gait assessments under single and dual task conditions at all study visits. The specific procedures for the PGS and single and dual task gait assessments are described in the Study Reference Manual.

Freezing of Gait (FOG)—Snijders protocol: FOG is a symptom where patients experience a paroxysmal inability to either continue or initiate gait (Shine et al., 2011). FOG is an independent risk factor for future falls in PD (Latt et al., 2009). Though dopaminergic pathways account for some of the pathophysiology, FOG is closely associated with gait disturbance, impaired executive function and attention, and diminished neocortical cholinergic innervation (Bohnen et al., 2014; Peterson et al., 2015; Shine et al., 2011). To elicit FOG in an experimental setting, several tests were performed to determine the most sensitive capture of FOG relative to a ‘gold standard’ FOG questionnaire (Snijders et al., 2012). A procedure involving four rapid, full 360-degree turns in both directions (i.e. two leftward full turns and two rightward full turns—in random order) was determined to be the most effective test to provoke FOG. This study will employ the same narrow quarter, four rapid 360-degree turns (two leftward and two rightward turn) in addition to dual task gait trajectory and normal gait trajectory explained in the Snijders protocol (Snijders et al., 2012) to elicit FOG. While subjects perform this assessment battery, they will wear the Opal sensors by APDM if available at the site, which will quantitatively assess a Freezing Ratio correlate of FOG.

The Movement Disorder Society Unified Parkinson's Disease Rating Scale Motor Section (MDS-UPDRS) Part III: The UPDRS is considered the ‘gold standard’ clinical scale for PD (Song et al., 2009). The MDS-UPDRS is a revision of the original UPDRS (Goetz et al., 2008). The motor section of the MDS-UPDRS (MDS-UPDRS-III) is composed of an 18-item section in which each parameter is rated on a zero to four scale, where four represents the worst disability and zero represents no disability. The Postural Instability and Gait Difficulty (PIGD) subscale is composed of 5 items: Section II of the MDS-UPDRS items 2.13-2.15 and section III items 3.29-3.30. PIGD score correlates highly with fall risk (Rudzinska et al., 2007) and deficits of executive function (Xu et al., 2014).

University of Pennsylvania Smell Identification Test (UPSIT): The olfactory system is a cholinergic rich area of the brain that degenerates early in PD (Doty, 2012; Mancini and Horak, 2010). Approximately 90% of patients with early stage PD have olfactory dysfunction and olfactory dysfunction correlates with loss of cholinergic neurons in structures including the nucleus basalis of Meynert (nBM). The UPSIT is a 40 item ‘scratch-and-sniff’ test that takes about 15 minutes to administer and is a reliable measure of olfactory function.

Safety and Screening Assessments: An AE is any untoward medical occurrence associated with the use of a drug in humans, whether or not considered drug related. Therefore an AE can be ANY unfavorable and unintended sign (including an abnormal laboratory finding or vital sign measurement), symptom, or disease temporally associated with the use of a medicinal product, without any judgment about causality.

Events meeting the definition of an AE include: Exacerbation of a chronic or intermittent pre-existing condition including either an increase in frequency and/or intensity of the condition. New conditions detected or diagnosed after investigational product administration even though it may have been present prior to the start of the study. Signs, symptoms, or the clinical sequelae of a suspected drug interaction. Signs, symptoms, or the clinical sequelae of a suspected overdose of either investigational product or a concomitant medication. Clinically significant abnormal findings (laboratory test results, vital signs, physical examination findings, ECGs, radiologic exams or other studies) should be recorded as AEs. A “clinically significant” finding is one that affects clinical management, including additional visits, monitoring or referrals, diagnostic tests or alteration of treatment, or that is considered clinically significant by the investigator. A clinically significant finding may be a change in a test that has previously been abnormal but now requires additional action. When a medical or surgical procedure is performed, the condition that leads to the procedure should be recorded as the AE.

Events that do not meet the definition of an AE include: Anticipated day-to-day fluctuations or expected progression of pre-existing disease(s) or condition(s) present or detected at the start of the study unless judged by the investigator to be more severe than expected for the subject's underlying condition. Abnormal laboratory, ECG, or vital sign measurements that are not labelled clinically significant (see definition above). Situations where an untoward medical occurrence did not occur (social and/or convenience admission to a hospital). Overdose in the absence of other AEs will not be reported as an AE in its own right. Changes in C-SSRS during the course of the study indicating worsening should be evaluated by the investigator for clinical significance, and if clinically significant (e.g., alteration in medical care or intervention is required), an associated AE should be recorded, if present. The AE should be the primary underlying clinical manifestation assessed as clinically significant, and not the change in score itself.

Adverse events are recorded from the time that informed consent is signed, including those that occur during the Single-Blind Run-in Period. Treatment emergent adverse events are defined as those that occur on or after the date of the first dose of investigational product.

Definition of Serious Adverse Event: An AE is considered serious if, in the view of either investigator or sponsor, it results in any of the following outcomes: Death, A life-threatening AE. An AE is considered “life-threatening” if, in the view of either the investigator or sponsor, its occurrence places the patient or subject at immediate risk of death. It does not include an AE that, had it occurred in a more severe form, might have caused death. The determination of whether an AE is life threatening can be based on the opinion of either the investigator or sponsor. Thus, if either believes that it meets the definition of life-threatening, it must be considered life-threatening for reporting purposes. Inpatient hospitalization or prolongation of existing hospitalization, A persistent or significant incapacity or substantial disruption of the ability to conduct normal life functions, or A congenital anomaly/birth defect.

Important medical events that may not result in death, be life threatening, or require hospitalization may be considered serious when, based upon appropriate medical judgment, they may jeopardize the patient or subject and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

This definition of an SAE permits either the sponsor or the investigator to decide if an event is serious. Because SAEs are critically important for the identification of significant safety problems, FDA believes taking into account both the investigator's and the sponsor's assessment is important. For example, the investigator's perspective may be informed by having actually observed the event, and the sponsor is likely to have broader knowledge of the drug and its effects to inform its evaluation of the significance of the event. If either the sponsor or investigator believes that the event is serious, the event must be considered serious and evaluated by the sponsor for possible expedited reporting.

Physical Examinations: Physical examinations will be performed as indicated in Table 3. A complete physical examination will include, at a minimum, assessment of the cardiovascular, respiratory, gastrointestinal, and neurological systems. Neurological examinations will include assessment of gait, balance, coordination, cranial nerves and motor and sensory systems. A brief, symptoms-directed physical examination will include, at a minimum, assessments of the lungs, cardiovascular system, and abdomen (liver and spleen). Physical examinations at Screening and Visit 8 will be full examinations; at all other study visits, an abbreviated physical examination is required.

Vital Signs: Vital signs will be measured after the subject has been in the seated position for 5 minutes and will include temperature, systolic and diastolic blood pressures, pulse rate, and respiratory rate. Postural changes will be measured within 3 minutes of appropriate body position change. Body weight will also be recorded at each visit and height will be recorded at Screening.

Electrocardiogram: Single 12-lead ECGs will be obtained at each time point during the study using an ECG machine that automatically calculates the heart rate and measures PR, QRS, QT, and QTc intervals with the subject in the supine position. The investigator or designated qualified physician at the site will evaluate the Screening ECG for any abnormalities that should exclude the subject from the study or require acute additional evaluation or intervention. They should also evaluate the ECG printouts for all subsequent visits for any new abnormalities. Any abnormality should include a determination of clinical significance. A clinically significant ECG finding is one that requires additional medical evaluation or treatment.

Clinical Safety Laboratory Assessments: All protocol-required laboratory assessments, as defined in Table 4, must be conducted in accordance with the Study Procedures Manual and Protocol Time and Events Schedule (Table 3). Laboratory requisition forms must be completed and samples must be clearly labelled with the subject number, protocol number, site/center number, and visit date. Details for the preparation and shipment of samples will be provided by the laboratory and are detailed in the Study Procedures Manual. Reference ranges for all safety parameters will be provided to the site by the laboratory responsible for the assessments. Abnormal laboratory tests that are clinically significant should also be recorded as AEs on the eCRF. Clinically significant means that the confirmed abnormal test result has an impact on patient management, including additional monitoring diagnostic tests, or changes in treatment. The same standard applies to additional non-protocol specified laboratory assessments that are performed at the institution's local laboratory and result in a change in subject management (i.e., monitoring, diagnostic tests, or any alteration in treatment). Refer to the Study Procedures Manual for appropriate processing and handling of samples to avoid duplicate and/or additional blood draws. Hematology, clinical chemistry, urinalysis, and other screening laboratory parameters to be tested are listed in Table 4. All laboratory tests with values that are considered clinically significantly abnormal during participation in the study or within 7 days after the last dose of investigational product should be repeated until the values return to normal or baseline or until the value stabilizes. If such values do not return to normal within a period judged reasonable by the investigator, the etiology should be identified and the Medical Monitor notified.

TABLE 4
Protocol-Required Screening and Safety Laboratory Assessments

| Laboratory | | | |
|---|---|---|---|
| Assessments | Parameters | | |
| Hematology | Platelet count | RBC Indices | WBC Count with |
| | RBC count | MCV | Differential |
| | Hemoglobin | MCH | Neutrophils |
| | Hematocrit | | Lymphocytes |
| | | | Monocytes |
| | | | Eosinophils |
| | | | Basophils |
| Clinical Chemistry | BUN | Potassium | |
| | Creatinine | Sodium | ALT |
| | Glucose | Calcium | Alkaline |
| | | Chloride | phosphatase |
| | | Bicarbonate | Total and |
| | | | direct bilirubin |
| | | | Total protein |
| | | | Albumin |
| | | | GGT |

| | |
|---|---|
| Routine Urinalysis | Specific gravity |
| | pH, glucose, protein, blood, and ketones by dipstick |
| | Microscopic examination (if blood or protein is |
| | abnormal) |
| Screening Tests | Drugs and alcohol screening |
| only | HBsAg |
| | Hepatitis C antibody |
| | TSH |
| | Vitamin B₁₂ |
| | Syphilis serology |
| | Serum or urine hCG pregnancy test (as needed for |
| | women of child bearing potential) |

Abbreviations:
ALT = alanine aminotransferase;
AST = aspartate aminotransferase;
BUN = blood urea nitrogen;
FSH = follicle stimulating hormone;
GGT = gamma glutamyltransferase;
HBsAg = hepatitis B surface antigen;
hCG = human chorionic gonadotropin;
MCH = mean corpuscular hemoglobin;
MCV = mean corpuscular volume;
RBC = red blood cell;
TSH = thyroid stimulating hormone;
WBC = white blood cell.

Efficacy Analyses: Efficacy data will be summarized and listed by treatment and assessment time by period and overall. The between treatment differences for the efficacy endpoints of interest will be estimated using a mixed effect model with sequence, period, and treatment as fixed effect and subject-within-sequence as random effect. The stratification variable and baseline efficacy value will be included as a covariate of interest. The least squares means, treatment difference and 95% CIs, P-values will be estimated for efficacy endpoints, including the following: Change in gait speed as measured on an electronic walkway system from the start to the end of each treatment period with intepirdine. Change in step time variability as measured on an electronic walkway system from the start to the end of each treatment period with intepirdine. Change in step length variability as measured on an electronic walkway system from the start to the end of each treatment period with intepirdine. Change in the mini-BESTest total score and change from baseline in measures of dynamic gait and balance assessed with Opal APDM sensors during the mini-BESTest evaluation from the start to the end of each treatment period with intepirdine. Change in freezing of gait score and freezing ratio using Opal APDM sensors while turning, from the start to the end of each treatment period with intepirdine. Change in MDS-UPDRS Parts II and III and PIGD sub-scores from the start to the end of each treatment period with intepirdine. Change from baseline in UPSIT score from the start to the end of each treatment period with intepirdine. The baseline for the efficacy analysis is defined as the start of each individual treatment period. Carry over effect will be tested and evaluated based on the baseline in each treatment period.

### Example 2

### A Phase 2, Double-Blind, Randomized, Placebo-Controlled Crossover Study Evaluating the Effect of Intepirdine on Gait and Balance in Patients with Dementia but not Experiencing Falls

Primary Objective: To assess the effect of intepirdine versus placebo on gait speed, a quantitative measure of functional mobility, evaluated under normal and dual-task walking conditions on an electronic walkway system after 2 weeks of treatment.

Secondary Objection: To assess the safety and tolerability of intepirdine.

Exploratory Objectives: To assess the effect of intepirdine versus placebo on gait variability, evaluated under normal and dual-task walking conditions on an electronic walkway system after 2 weeks of treatment; to assess the effect of intepirdine versus placebo on gait and balance, as measured by the mini-BESTest battery, after 2 weeks of treatment; to assess the effect of intepirdine versus placebo on gait and balance, as measured by individual item scores calculated by Opal APDM sensors during performance of the mini-BESTest battery, after 2 weeks of treatment; to assess the effect of intepirdine versus placebo on freezing of gait (FOG), after 2 weeks of treatment; to assess the effect of intepirdine versus placebo on movement and balance, as measured by the Motor Disorder Society Unified Parkinson's Disease Rating Scale (MDS-UPDRS) Part III and PIGD subscore, after 2 weeks of treatment; and to assess the effect of intepirdine versus placebo on smell, a proxy for cholinergic function, as measured by the University of Pennsylvania Smell Identification Test (UPSIT), after 2 weeks of treatment.

Approximately 30-40 patients with dementia (AD, DLB, or PDD) and gait impairment will be enrolled. Subjects must be on a stable regimen of a cholinesterase inhibitor for at least two months prior to screening, and must have received a cholinesterase inhibitor for at least four months. Subjects may be on certain other background therapies if on a stable regimen prior to screening. These allowed background therapies and stability requirements are listed in Table 1.

Key Inclusion Criteria: Subjects eligible for enrollment in the study must meet all of the following criteria: Male or female subject with a clinical diagnosis of Alzheimer's disease (AD), dementia with Lewy bodies (DLB), or Parkinson's disease dementia (PDD). The diagnosis of AD must be in accordance with the recommendations from the National Institute on Aging-Alzheimer's Association workgroups on diagnostic guidelines for AD (McKhann et al., 2011). The diagnosis of PDD must be in accordance with the Movement Disorder Society (MDS) criteria for PDD (Emre et al., 2007). The diagnosis of DLB must be in accordance with Consensus criteria for DLB diagnosis (McKeith et al., 2005). Subject has an MMSE score 18 to 26 inclusive, at both the Screening and Baseline Visits. Subject must have experienced less than one fall within the past month as assessed by history gathered by the clinical investigator at the time of the Screening Visit. Clinically relevant risk of fall. For example, patients have an average gait speed of less than 100 cm/s on an electronic walkway evaluation or abnormal gait and balance on the miniBEST assessment with a score less than 16. In case these criteria are not met, a justification for the clinically determined fall risk should be documented and discussed with the medical monitor. Other documented risks of fall or gait impairment may be acceptable with approval from the medical monitor. Mandatory need for a walking aid could for example be considered as such a condition. Subject has a Hachinski Ischaemia score less than or equal to 4 at Screening. Subject has a documented history of at least 6 weeks of stable cholinesterase inhibitor therapy prior to screening with no intent to change for the duration of the study. If the subject is receiving dopaminergic therapy for parkinsonism or other motor impairment, the subject must have a documented history of at least 60 days of therapy at a stable dose prior to the Screening Visit and there is no intent to change the dose for the duration of the study. Age 50 to 89 years, inclusive, at the time of the Screening Visit. Female subjects must be: Of non-childbearing potential (i.e., any female who is post-menopausal [greater than 1 year without menstrual period in the absence of hormone replacement therapy]) or surgically sterile; or, If pre-menopausal or menopausal for 1 year or less, must have a negative pregnancy test and must not be lactating at the Screening and Baseline Visits. Female subjects of childbearing potential and who are sexually active are required to practice adequate methods of birth control. Female subjects for whom menopausal status is in doubt in the opinion of the investigator will be required to use an adequate form of birth control. Acceptable, adequate form of birth control is defined as consistent use of combined effective methods of contraception including at least one barrier method. Male subjects who are sexually active are also be required to use an adequate form of birth control as described above. Subject has the ability to comply with procedures for evaluating gait, balance, and other testing in the opinion of the investigator. Subject must be able to ingest pills (in tablet form) whole; Subject lives with (or has substantial periods of contact with) a regular caregiver or healthcare provider who is willing to attend visits, oversee the subject's compliance with protocol-specified procedures and study medication, and report on subject's status, and who has substantial contact with the subject. If the caregiver does not cohabitate with the subject, he/she ideally should have a minimum of 10 hours total and at least 3 days contact with the subject per week. Prior to randomization, study representatives will review eligibility of non-cohabitating caregivers. Every effort should be made to have the same caregiver/healthcare provider throughout the study; Subject has provided full written informed consent prior to the performance of any protocol specified procedure; or if unable to provide informed consent due to cognitive status, subject has provided assent and a legally acceptable representative has provided full written informed consent on behalf of the subject; Caregiver has provided full written informed consent on his/her own behalf prior to the performance of any protocol-specified procedure; General health status is acceptable for participation for the entire duration of the study.

Key Exclusion Criteria: A subject will not be eligible for inclusion in this study if any of the following criteria apply: Other Causes for Dementia: History and/or evidence of any other CNS disorder that could be interpreted as a cause of dementia (in the opinion of the investigator), e.g., cerebrovascular disease (transient ischemic attack, stroke, hemorrhage); structural or developmental abnormality; epilepsy; infectious, degenerative, or inflammatory/demyelinating CNS conditions; Evidence of the following disorders where this is thought to be the cause of, or to contribute to the severity of, the subject's dementia: current vitamin B12 deficiency, hypothyroidism, neurosyphilis, or Wernicke's encephalopathy. Atypical clinical features or clinical course of dementia that would lead the investigator to conclude symptoms are more likely due to an alternate dementia diagnosis other than AD, DLB, or PDD. Confounding Medical Conditions: History of significant psychiatric illness such as schizophrenia or bipolar affective disorder or any other significant psychiatric illness that in the opinion of the investigator would interfere with participation in the study; history of major depressive disorder in the past year, or current major depressive episode. Significant suicide risk as defined by (1) suicidal ideation as endorsed on items 4 or 5 on the C-SSRS within the past year, at Screening or at Baseline; (2) suicidal behaviors within the past year; (3) clinical assessment of significant suicidal risk during subject interview. Current psychosis that in the opinion of the investigator would interfere with the subject's ability to participate in this study. History of epilepsy or unexplained seizure in the past 5 years, unexplained recent loss of consciousness, or history of significant head trauma with loss of consciousness. History of malignancy during the 5 years before Screening. History of basal cell carcinoma and melanoma in situ are permitted. History of other cancers currently in a non-active state may be acceptable after review with the Medical Monitor. Any clinically relevant concomitant disease including progressive liver or kidney dysfunction, history of myocardial infarction or unstable angina within 6 months of Screening, history of more than 1 myocardial infarction within 5 years of Screening, history of clinically significant stroke, or any other medical or psychiatric condition, which, in the opinion of the investigator, makes the subject unsuitable for inclusion in the study. History of alcohol use disorder or other substance abuse disorder (excluding tobacco use), according to the Diagnostic and Statistical Manual of Mental Disorders, 5th Edition, criteria in the past 10 years. History of Down syndrome or mental retardation. Severe and/or debilitating arthritis or peripheral neuropathy. Peripheral neuropathy should be assessed quantitatively with a 128-Hz clanging tuning fork test applied to the hallux of both feet. Vibration sensation by the subject for 4 seconds or less will be excluded due to severe neuropathy. Significant tremor which would interfere with recording balance and walking. Uncorrected vision disturbance or vestibular problems that would affect gait and balance. Symptomatic bradycardia which in the opinion of the investigator may place the subject at risk of syncope. Concomitant Medications: Participation in another investigational drug or device study in dementia during the 60 days prior to the Screening Visit (Visit 1), or within 5 half-lives of use of the investigational drug prior to the Screening Visit, whichever is longer. In addition, subjects who were previously screened for another study in dementia but failed the entry criteria for that study may be screened with no time delay prior to the Screening Visit, provided that, in the opinion of the investigator, and after review with the Medical Monitor, there is a realistic possibility that the subject would be eligible.

Permitted Medications and Non-Drug Therapies: All concomitant medications and those taken within 6 months prior to Screening, including over-the-counter and herbal remedies, will be recorded in the eCRF. Non-medication therapies related to the subject's dementia (e.g. neurostimulation, cognitive rehabilitation) that have occurred in the 12 months prior to Screening must also be recorded. The name of the drug, the dose, indication and route of administration as well as the dates administered should be documented; the minimum requirement is to record the drug name and dates of administration. Any medication not specified in the list of prohibited and conditional medications provided in Table 1 is permitted during the study. Treatment with any concomitant medications as detailed in Table 1, unless otherwise specified, need to have been discontinued for 5 half-lives prior to screening and assessed as no longer clinically necessary for the subject.

Unacceptable Test/Laboratory Values: Persistent or recurrent liver enzyme elevations, alanine transaminase (ALT) and/or aspartate aminotransferase (AST) greater than or equal to 2.0 times upper limit of normal (ULN). Total bilirubin over 1.5×ULN except due to documented Gilbert's disease. Calculated creatinine clearance less than 40 mL/min (Cockroft-Gault formula): Adult males: [(140—age in years)×(weight in kg)]±72×serum creatinine* Adult females: 0.85×[((140—age in years)×(weight in kg))±72×serum creatinine*]*in mg/dL. Positive hepatitis B surface antigen or hepatitis C antibody test. Confirmed corrected QT interval (QTc) value greater than or equal to 450 msec for males or greater than or equal to 470 msec for females. Subjects with a QRS value greater than 120 msec and QTc value less than 500 msec may be eligible following discussion with the Medical Monitor. Clinically relevant renal dysfunction: plasma creatinine >1.5 mg/dl, when considered clinically significant.

Other ineligibility criteria: Previous exposure to intepirdine or SB742457. Subject is unable to take study medication as prescribed throughout the study (with assistance is acceptable), has a significant history of non-compliance with prescribed medication, or is at risk of non-compliance with study medication or procedures. Subject or caregiver is an immediate family member or employee of the participating investigator, any of the participating site staff, or of the sponsor study staff. Subject was prescribed cognitive tasks for cognitive rehabilitation performed under medical supervision in the 3 months prior to Screening and/or during the study. Subject has participated in a program of neurostimulation in the past 3 months or plans to participate in a program of neurostimulation during the course of the study.

Investigational Product (IP) and Other Study Treatment: IP in this study is defined as 35 mg intepirdine tablets and their matching placebo, and will be provided by Axovant Sciences. intepirdine and placebo tablets will be indistinguishable from each other. All subjects will take one tablet of IP daily at bedtime, with or without food; this will consist of either one tablet of 15 mg intepirdine or one tablet of placebo. See Table 5.

IP for the Single-Blind Run-In Period (Weeks −2 to 0) will be supplied at Visit 2. Subjects will be given an IP kit containing 1 bottle of placebo tablets. Each bottle will contain 50 tablets, which is sufficient medication for 2 weeks plus 36 days overage. Subjects will be instructed to take one tablet of IP daily at bedtime with or without food. In the event that the Single-Blind Run-In period is extended, no new IP will be dispensed. Drug accountability will be checked, and subjects will be re-dispensed the study medication kit and bottles they received at Visit 2.

IP for the Double-Blind Treatment Period (Weeks 0 to 8) will be supplied at Visits 3, 4, and 5. At each dispensing visit during the Treatment Period, subjects will be given an IP kit containing one bottle of IP. Depending on treatment assignment, subjects will receive either one bottle of 15 mg intepirdine tablets or one bottle of placebo tablets. Each bottle will contain 50 tablets, which is sufficient for 4 weeks plus 22 days overage. Subjects will be instructed to take one tablet of IP daily at bedtime with or without food. New bottles of IP will not be dispensed at Visit 6, however, drug accountability will be checked. All subjects and their caregivers should be instructed to bring IP bottles, with any unused drug, to each visit.

TABLE 5
Investigational Product and Other Study Treatment

| | | |
|---|---|---|
| Product name: | Investigational | Placebo |
| | Product | |
| | intepirdine | |
| Formulation description: | pink, film-coated, | pink, film-coated, |
| | round tablets | round tablets |
| Dosage form: | 15 mg Tablet | Placebo Tablet |
| Unit dose strength(s)/ | 15 mg | N/A |
| Dosage level(s): | 15 mg | placebo |
| Route of Administration: | Oral | Oral |
| Duration (Run-in Period): | N/A | 2 weeks |
| Duration (Treatment Period): | 2 weeks | 2 weeks |
| Dosing instructions: | Take before | Take before |
| | bedtime with or | bedtime with or |
| | without food | without food |
| Manufacturer/Source of | Metrics Contract | Metrics Contract |
| Procurement | Services | Services |
| | Greenville, NC | Greenville, NC |

Randomization/Treatment Assignment: FIG. 1 graphically depicts the study design. Subjects will be assigned to receive 15 mg RVT 101 or placebo in accordance with the randomization schedule, prior to the start of the Double-Blind Treatment Period, using validated software. Following confirmation of eligibility at Visit 3 (end of Single-Blind Run-In Period), subjects will be randomized to placebo or 15 mg RVT 101 in a 1:1 ratio. Each site will have an approximately 1:1 ratio of patients randomized to intepirdine and placebo.

Blinding: This will be a double-blind study. The study will include a 2-week Single-Blind Run-In Period during which investigators will know that the subject is taking placebo but the subject/caregiver will not. This will be followed by a 6-week Double-Blind Treatment Period consisting of three parts: a 2-week Treatment Period B, a 2-week Washout Period, and a 2-week Crossover Treatment Period C. During the Double-Blind Treatment Period, when neither subjects nor investigators will know which of the two treatments the subject is receiving. Subjects and caregivers will not be informed of transition from the Single-Blind Run-In Period to the Double-Blind Treatment Period. RVT 101 and placebo will be provided as tablets that are indistinguishable in appearance, smell, and taste.

The following will apply: The investigator or treating physician may unblind a subject's treatment assignment. Such a measure should be taken only in the case of an emergency OR in the event of a serious medical condition when knowledge of the investigational product is essential for the appropriate clinical management or welfare of the subject as judged by the investigator. In the event that a medical emergency or condition requires knowledge of the subject's treatment assignment, the investigator will contact the medical monitor to discuss the potential need for unblinding. The procedure of unblinding for a specific subject is provided in the Study Reference Manual. If, for safety reasons, the investigator determines that the subject's treatment must be immediately unblinded to provide adequate medical treatment, the investigator must inform the Medical Monitor about the unblinding as soon as possible, but without revealing the treatment assignment of the unblinded subject. The sponsor will be informed without delay of the decision to unblind any subject and will determine whether any additional measures need to be taken for the safety of subjects currently in the study. Any other requests to reveal a subject's treatment identity must be requested of, and approved by, Axovant Sciences. A subject will be withdrawn from the study if his or her treatment code is unblinded by the investigator or treating physician. The date and reason for the unblinding must be fully documented in the eCRF. Axovant Sciences or their designee may unblind the treatment assignment for any subject if this is required to fulfill regulatory reporting obligations such as expedited SAE reporting.

Time and Events: The Time and Events Schedule (Table 3) displays each study assessment and procedure along with the time of occurrence. All study assessments should be conducted by the investigator, and/or a suitably qualified designee approved and documented for this study. All raters will be trained and certified to perform the specific rating scales in this study.

Visit 3 will occur within 14 to 17 days after Visit 2. For Visits 4 through 6, there is a visit window of +3 days. If the visit window is used, the subsequent visit should be scheduled according to the date when the prior study visit actually took place and not according to the original visit schedule (i.e., the subsequent visit date should be re-calculated from the date of the previous visit). Information will be recorded in the source documents and, where appropriate, the case report form. If, during the visit, the subject is unable or, in the judgment of the investigator, unlikely to be able to complete the cognitive assessments, the testing may be rescheduled within the windows described previously. Subjects may be given breaks during the assessments or may have assessments split across different days so long as all assessments are completed within the visit window. Individual assessments should, however, be completed within a single day. Assessment should occur in the following order whenever possible: MMSE (screening only), Modified Hachinski Ischemia Scale (mHIS, screening only), motor testing (electronic walkway tests, miniBESTest, FOG assessment, for patients with Lew Body dementia who are in the “off” state or show motor fluctuations, motor testing should be done during the “on” phase), caregiver FES (Screening only, UPSIT, C-SSRS, MDS-UPDRS, physical and neurological exam, 12-lead ECG, vital signs, blood draws.

Screening Period (up to 28 days before Visit 2): Subjects will be screened for eligibility during the Screening Period. Subjects who do not qualify for the study during this period will be considered screen failures. An ICF will be signed by each subject, if they are able, or by the caregiver with subject assent. An ICF will also be signed by the caregiver before any study-specific procedures are performed. Subjects will be screened according to study inclusion/exclusion criteria. This Screening Period may be extended for up to an additional 14 days if needed to complete assessment activities after approval by the study Medical Monitor. Subjects who are screen failures during the Screening Period may be rescreened after discussion with the Medical Monitor. Note: Subjects who are screen failures may be rescreened only once.

At the screening visit, all potential subjects will undergo MMSE assessment and gait speed evaluation on an electronic walkway system. If possible, the MMSE should be the first assessment performed, followed by the electronic walkway assessment. If the MMSE score or gait speed do not meet the criterion for randomization at screening, no other assessments should be performed.

Single-Blind Run-In Period (14+5 days before Visit 3): At Visit 2, subjects who meet all study screening criteria will enter a Single-Blind Run-In Period. Investigational product will be dispensed. Subjects will be instructed to take the investigational product once daily in the morning. Subjects will be instructed to take the first Single-Blind Run-In investigational product (single-blind placebo) during the study visit. Visit 2 assessments will be performed according to Table 3. To qualify for randomization at Baseline (Visit 3) subjects must return unused study medication, be considered capable of completing study assessments, remain within study-specified criteria for MMSE, meet criteria for having slow gait as described in the protocol (i.e. gait speed below 100 cm/s on electronic walkway analysis), and meet all other eligibility requirements.

The Single-Blind Run-In Period may be extended for up to 3 weeks after discussion with the Medical Monitor for subjects who do not meet the gait speed threshold necessary for randomization at Visit 3, and may be re-evaluated one additional time during this extension to determine eligibility. This subsequent visit will then serve as the subject's baseline if randomization criteria are met. No new investigational product will be dispensed for the additional run-in period; subjects should be instructed to continue taking blinded investigational product dispensed at Visit 2. The MMSE should be the first assessment performed, followed by the electronic walkway assessment, at the baseline visit. If the MMSE score or gait speed do not meet the criterion for randomization at Visit 3, no other assessments should be performed. If after discussion with the Medical Monitor it is determined that the Single-Blind Run-In Period may be extended by 3 weeks per the criterion above, the subject should be scheduled to return to the clinic in 3 weeks' time (+3 days), and all baseline assessments should be performed at this repeat visit.

Baseline (Visit 3) and Double-blind Treatment (Period B) (Visit 3-Visit4): At Visit 3 (Baseline), assessments will be performed to determine subject eligibility. Eligible subjects will be randomized 1:1 to one of the following treatment sequences: AP=intepirdine in Period B and placebo in Period C; PA=placebo in Period B and intepirdine in Period C. During this double-blind treatment period (Period B), investigational product will be dispensed at Visit 3. Subjects will ingest the first dose of investigational product in the clinic in the presence of study personnel. All additional doses will be ingested as outpatients. Subjects who prematurely discontinue double-blind investigational treatment in Period B should be encouraged to return to the clinic for an Early Termination Visit, and Visit 7 assessments and procedures will be completed.

Washout Period (Visit 4-Visit5): No investigational product will be given during the washout period. After the two-week washout, subjects will enter the second two-week treatment period (Period C). This period has a 5 day allowable window to accommodate scheduling conflicts. Subjects who prematurely discontinue from the washout period should be encouraged to return to the clinic for an Early Termination Visit, and Visit 7 assessments and procedures will be completed.

Double-blind Treatment (Period C) (Visit 5-Visit6): During the second two-week double-blind treatment period (Period C), subjects who received intepirdine during Period B will receive placebo; subjects who received placebo during Period B will receive intepirdine. During this double-blind treatment period (Period C), investigational product will be dispensed at Visit 5. Subjects will ingest the first dose of investigational product in the clinic in the presence of study personnel. All additional doses will be ingested as outpatients. Subjects who prematurely discontinue double-blind investigational treatment in Period C should be encouraged to return to the clinic for an Early Termination Visit, and Visit 7 assessments and procedures will be completed.

Critical Baseline Assessments: All baseline and efficacy assessments should be performed following the methodology included in the study manual. The procedures are briefly summarized below.

Mini-Mental State Evaluation (MMSE): The MMSE (Folstein et al., 1975) consists of 11 tests of orientation, memory (recent and immediate), concentration, language, and praxis. Scores range from 0 to 30, with lower scores indicating greater cognitive impairment. It is based on the performance of the subject and takes approximately 5 to 10 minutes to administer. The MMSE for patients included in the study must be between 14 and 26 inclusive at both the screening and baseline visits in order to qualify for randomization into the study.

Gait Speed Measured on Electronic Walkway Assessment: Electronic walkway systems, such as the GAITRite system, are computerized assessment tools that utilize an electronic walkway mat consisting of pressure-sensitive pads that can calculate spatiotemporal gait parameters including gait velocity, cadence, step time, step length, stride length, stride time, swing time, stance time, and double support time (Buesing et al., 2015). Subjects included in the study must have an average gait speed of less than 100 cm/s, or other clinically relevant risk of falls at Visits 1, 2 and 3. Subjects without assistive devices will undergo the Primary Gait Screen assessment (PGS) at Visits 1, 2 and 3, and all subjects will complete additional gait assessments under single and dual task conditions at all study visits. The specific procedures for the PGS and single and dual task gait assessments, including the calculation to determine study eligibility, are described in the Study Reference Manual.

University of Pennsylvania Small Identification Test (UPSIT): The olfactory system is a cholinergic rich area of the brain that degenerates early in PD. Approximately 90% of patients with early stage PD have olfactory dysfunction, and olfactory dysfunction correlates with loss of cholinergic neurons in structures such as the nucleus basalis of Meynert (nBM). The UPSIT is a 40 item ‘scratch-and sniff’ test that takes about 15 minutes to administer and is a reliable measure of olfactory function. The materials for the test will be provided to the sites, and the procedure for administering the test are described in the Study Reference Manual.

Caregiver-adapted version of the Fall Efficacy Scale: This scale will be rated by the caregiver and will capture his/her degree of concern about the likelihood of a patient falling while under a variety of different circumstances and performing different activities. The scale is adapted from the Falls Efficacy Scale—International, which is a 16-item self-reported scale in which individuals rate their degree of concern regarding the likelihood of their falling during different activities. The degree of concern is reflected in a 1-4 rating for each item, with “1” indicating that the individual is “not at all concerned” and “4” indicating that the individuals is “very concerned.” The scale has been reported to have strong internal validity (Cronbach's alpha=0.96) and test-retest reliability (ICC=0.96) (Yardley et al.,2005). A caregiver-adapted version of this scale will be used in this study and administered only at the screening visit.

Handgrip Strength Assessment: Low handgrip strength is a clinically relevant indicator of poor mobility ((Cruz-Jentoft et al., 2010). Moreover, baseline handgrip strength has been shown to have a linear relationship with risk of incident disability of activities of daily living (Al Snih et al., 2004). In this study, handgrip strength will be captured at screening. Handgrip strength will be assessed by a baseline hand dynamometer embedded with a strain gauge, as described on the Study Reference Manual.

Modified Hachinski Ischemia Scale (mHIS): This scale asks about the presence or absence of 8 clinical symptoms. It is being used in this study at Screening to identify and exclude subjects with vascular dementia.

Efficacy Assessments: All study assessments should be conducted by the investigator, and/or a suitably qualified designee, all of whom will be trained and certified to administer these measures for this study. Every effort should be made for the same person to conduct specific assessments on each individual subject at each study visit. Assessments may be monitored for quality. Screening assessments along with accompanying data will be reviewed to ensure that subjects meet the inclusion criteria. Other assessments will be monitored by using data collected.

Mini Balance Evaluation Systems Test (Mini-BESTest): The Mini-BESTest is a short, well-validated 14 item assessment of dynamic balance, specifically anticipatory postural transitions, postural responses, and dynamic gait (King et al., 2012). Each item is scored from (0-2) with a score of 2 being normal. The total score is out of 28. This test has excellent test-retest reliability (ICC=0.96) and interrater reliability (ICC=0.96) (Godi et al., 2013). Another advantage of the Mini-BESTest is its dual task walking assessment which is a reliable test of attention and executive function (Yarnall et al., 2011). The test takes about 10 minutes to administer (Mancini and Horak, 2010). The Mini-BESTest assessment will be performed with subjects wearing the Opal APDM sensors if available at the site, which will collect quantitative measures of dynamic gait and balance.

Electronic Walkway Assessment: Electronic walkway systems, such as the GAITRite system, are computerized assessment tools that utilize an electronic walkway mat consisting of pressure-sensitive pads that can calculate spatiotemporal gait parameters including gait velocity, cadence, step time, step length, stride length, stride time, swing time, stance time, and double support time (Buesing et al., 2015). Parameters such as gait velocity and cadence on the electronic walkway system are highly correlated with manually timed tests in PD patients (ICC=0.96, p<0.0005) (Bryant et al., 2013). Gait variability, specifically step length and step time, predict falls (Hausdorff, 2005) (Bryant et al., 2013). Stride length has been highly correlated with the PIGD-UPDRS subscore (see 6.3.1.3) (r=−0.90, p<0.0001) (Salarian et al., 2004). Further, electronic walkway systems are highly sensitive to demonstrating the effects of medications used to treat PD. The GAITRite system is able to detect gait changes with dopaminergic medications only 45 minutes after administration (Bryant et al., 2013). Recently, evidence has shown that cholinesterase inhibitors are also able to improve gait parameters in AD and PD (Henderson et al., 2016). Hence, the parameters measured by an electronic walkway system correlate well with established clinical scoring methods of gait, and these parameters are reliable and sensitive to capturing the effects of pharmacologic treatment. Subjects without assistive devices will undergo the Primary Gait Screen assessment (PGS) at Visits 1, 2 and 3, as well as additional gait assessments under single and dual task conditions at all study visits. The specific procedures for the PGS and single and dual task gait assessments are described in the Study Reference Manual.

Freezing of Gait (FOG)—Snijders protocol: FOG is a symptom where patients experience a paroxysmal inability to either continue or initiate gait (Shine et al., 2011). FOG is an independent risk factor for future falls in PD (Latt et al., 2009). Though dopaminergic pathways account for some of the pathophysiology, FOG is closely associated with gait disturbance, impaired executive function and attention, and diminished neocortical cholinergic innervation (Bohnen et al., 2014; Peterson et al., 2015; Shine et al., 2011). To elicit FOG in an experimental setting, several tests were performed to determine the most sensitive capture of FOG relative to a ‘gold standard’ FOG questionnaire (Snijders et al., 2012). A procedure involving four rapid, full 360-degree turns in both directions (i.e. two leftward full turns and two rightward full turns—in random order) was determined to be the most effective test to provoke FOG. This study will employ the same narrow quarter, four rapid 360-degree turns (two leftward and two rightward turn) in addition to dual task gait trajectory and normal gait trajectory explained in the Snijders protocol (Snijders et al., 2012) to elicit FOG. While subjects perform this assessment battery, they will wear the Opal sensors by APDM if available at the site, which will quantitatively assess a Freezing Ratio correlate of FOG.

The Movement Disorder Society Unified Parkinson's Disease Rating Scale Motor Section (MDS-UPDRS) Part III: The UPDRS is considered the ‘gold standard’ clinical scale for PD (Song et al., 2009). The MDS-UPDRS is a revision of the original UPDRS (Goetz et al., 2008). The motor section of the MDS-UPDRS (MDS-UPDRS-III) is composed of an 18-item section in which each parameter is rated on a zero to four scale, where four represents the worst disability and zero represents no disability. The Postural Instability and Gait Difficulty (PIGD) subscale is composed of 5 items: Section II of the MDS-UPDRS items 2.13-2.15 and section III items 3.29-3.30. PIGD score correlates highly with fall risk (Rudzinska et al., 2007) and deficits of executive function (Xu et al., 2014).

University of Pennsylvania Smell Identification Test (UPSIT): The olfactory system is a cholinergic rich area of the brain that degenerates early in PD (Doty, 2012; Mancini and Horak, 2010). Approximately 90% of patients with early stage PD have olfactory dysfunction and olfactory dysfunction correlates with loss of cholinergic neurons in structures including the nucleus basalis of Meynert (nBM). The UPSIT is a 40 item ‘scratch-and-sniff’ test that takes about 15 minutes to administer and is a reliable measure of olfactory function.

Safety and Screening Assessments: An AE is any untoward medical occurrence associated with the use of a drug in humans, whether or not considered drug related. Therefore an AE can be ANY unfavorable and unintended sign (including an abnormal laboratory finding or vital sign measurement), symptom, or disease temporally associated with the use of a medicinal product, without any judgment about causality.

Events meeting the definition of an AE include: Exacerbation of a chronic or intermittent pre-existing condition including either an increase in frequency and/or intensity of the condition. New conditions detected or diagnosed after investigational product administration even though it may have been present prior to the start of the study. Signs, symptoms, or the clinical sequelae of a suspected drug interaction. Signs, symptoms, or the clinical sequelae of a suspected overdose of either investigational product or a concomitant medication. Clinically significant abnormal findings (laboratory test results, vital signs, physical examination findings, ECGs, radiologic exams or other studies) should be recorded as AEs. A “clinically significant” finding is one that affects clinical management, including additional visits, monitoring or referrals, diagnostic tests or alteration of treatment, or that is considered clinically significant by the investigator. A clinically significant finding may be a change in a test that has previously been abnormal but now requires additional action. When a medical or surgical procedure is performed, the condition that leads to the procedure should be recorded as the AE.

Events that do not meet the definition of an AE include: Anticipated day-to-day fluctuations or expected progression of pre-existing disease(s) or condition(s) present or detected at the start of the study unless judged by the investigator to be more severe than expected for the subject's underlying condition. Abnormal laboratory, ECG, or vital sign measurements that are not labelled clinically significant (see definition above). Situations where an untoward medical occurrence did not occur (social and/or convenience admission to a hospital). Overdose in the absence of other AEs will not be reported as an AE in its own right. Changes in C-SSRS during the course of the study indicating worsening should be evaluated by the investigator for clinical significance, and if clinically significant (e.g., alteration in medical care or intervention is required), an associated AE should be recorded, if present. The AE should be the primary underlying clinical manifestation assessed as clinically significant, and not the change in score itself.

Adverse events are recorded from the time that informed consent is signed, including those that occur during the Single-Blind Run-in Period. Treatment emergent adverse events are defined as those that occur on or after the date of the first dose of investigational product.

Definition of Serious Adverse Event: An AE is considered serious if, in the view of either investigator or sponsor, it results in any of the following outcomes: Death, A life-threatening AE. An AE is considered “life-threatening” if, in the view of either the investigator or sponsor, its occurrence places the patient or subject at immediate risk of death. It does not include an AE that, had it occurred in a more severe form, might have caused death. The determination of whether an AE is life threatening can be based on the opinion of either the investigator or sponsor. Thus, if either believes that it meets the definition of life-threatening, it must be considered life-threatening for reporting purposes. Inpatient hospitalization or prolongation of existing hospitalization, A persistent or significant incapacity or substantial disruption of the ability to conduct normal life functions, or A congenital anomaly/birth defect.

Important medical events that may not result in death, be life threatening, or require hospitalization may be considered serious when, based upon appropriate medical judgment, they may jeopardize the patient or subject and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

This definition of an SAE permits either the sponsor or the investigator to decide if an event is serious. Because SAEs are critically important for the identification of significant safety problems, FDA believes taking into account both the investigator's and the sponsor's assessment is important. For example, the investigator's perspective may be informed by having actually observed the event, and the sponsor is likely to have broader knowledge of the drug and its effects to inform its evaluation of the significance of the event. If either the sponsor or investigator believes that the event is serious, the event must be considered serious and evaluated by the sponsor for possible expedited reporting.

Physical Examinations: Physical examinations will be performed as indicated in Table 3. A complete physical examination will include, at a minimum, assessment of the cardiovascular, respiratory, gastrointestinal, and neurological systems. Neurological examinations will include assessment of gait, balance, coordination, cranial nerves and motor and sensory systems. A brief, symptoms-directed physical examination will include, at a minimum, assessments of the lungs, cardiovascular system, and abdomen (liver and spleen). Physical examinations at Screening and Visit 8 will be full examinations; at all other study visits, an abbreviated physical examination is required.

Vital Signs: Vital signs will be measured after the subject has been in the seated position for 5 minutes and will include temperature, systolic and diastolic blood pressures, pulse rate, and respiratory rate. Postural changes will be measured within 3 minutes of appropriate body position change. Body weight will also be recorded at each visit and height will be recorded at Screening.

Electrocardiogram: Single 12-lead ECGs will be obtained at each time point during the study using an ECG machine that automatically calculates the heart rate and measures PR, QRS, QT, and QTc intervals with the subject in the supine position. The investigator or designated qualified physician at the site will evaluate the Screening ECG for any abnormalities that should exclude the subject from the study or require acute additional evaluation or intervention. They should also evaluate the ECG printouts for all subsequent visits for any new abnormalities. Any abnormality should include a determination of clinical significance. A clinically significant ECG finding is one that requires additional medical evaluation or treatment.

Clinical Safety Laboratory Assessments: All protocol-required laboratory assessments, as defined in Table 4, must be conducted in accordance with the Study Procedures Manual and Protocol Time and Events Schedule (Table 3). Laboratory requisition forms must be completed and samples must be clearly labelled with the subject number, protocol number, site/center number, and visit date. Details for the preparation and shipment of samples will be provided by the laboratory and are detailed in the Study Procedures Manual. Reference ranges for all safety parameters will be provided to the site by the laboratory responsible for the assessments. Abnormal laboratory tests that are clinically significant should also be recorded as AEs on the eCRF. Clinically significant means that the confirmed abnormal test result has an impact on patient management, including additional monitoring diagnostic tests, or changes in treatment. The same standard applies to additional non-protocol specified laboratory assessments that are performed at the institution's local laboratory and result in a change in subject management (i.e., monitoring, diagnostic tests, or any alteration in treatment). Refer to the Study Procedures Manual for appropriate processing and handling of samples to avoid duplicate and/or additional blood draws. Hematology, clinical chemistry, urinalysis, and other screening laboratory parameters to be tested are listed in Table 4. All laboratory tests with values that are considered clinically significantly abnormal during participation in the study or within 7 days after the last dose of investigational product should be repeated until the values return to normal or baseline or until the value stabilizes. If such values do not return to normal within a period judged reasonable by the investigator, the etiology should be identified and the Medical Monitor notified.

Efficacy Analyses: Efficacy data will be summarized and listed by treatment and assessment time by period and overall. The between treatment differences for the efficacy endpoints of interest will be estimated using a mixed effect model with sequence, period, and treatment as fixed effect and subject-within-sequence as random effect. The stratification variable and baseline efficacy value will be included as a covariate of interest. The least squares means, treatment difference and 95% CIs, P-values will be estimated for efficacy endpoints, including the following: Change in gait speed as measured on an electronic walkway system from the start to the end of each treatment period with intepirdine. Change in step time variability as measured on an electronic walkway system from the start to the end of each treatment period with intepirdine. Change in step length variability as measured on an electronic walkway system from the start to the end of each treatment period with intepirdine. Change in the mini-BESTest total score and change from baseline in measures of dynamic gait and balance assessed with Opal APDM sensors during the mini-BESTest evaluation from the start to the end of each treatment period with intepirdine. Change in freezing of gait score and freezing ratio using Opal APDM sensors while turning, from the start to the end of each treatment period with intepirdine. Change in MDS-UPDRS Parts II and III and PIGD sub-scores from the start to the end of each treatment period with intepirdine. Change from baseline in UPSIT score from the start to the end of each treatment period with intepirdine. The baseline for the efficacy analysis is defined as the start of each individual treatment period. Carry over effect will be tested and evaluated based on the baseline in each treatment period.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience, patentability, or both. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

What is claimed:

1. A method of improving the balance, gait, or both in a subject in need thereof, wherein the subject has a neurodegenerative disease and has not experienced an increase in falls, comprising administering to said subject a therapeutically effective amount of intepirdine, or pharmaceutically acceptable salts, hydrates or solvates thereof.

2. The method of claim 1, wherein the subject has experienced less than one fall per week prior to the administering of the therapeutically effective amount of intepirdine, or pharmaceutically acceptable salts, hydrates or solvates thereof.

3. The method of claim 1, wherein the subject has experienced less than one fall per month prior to the administering of the therapeutically effective amount of intepirdine, or pharmaceutically acceptable salts, hydrates or solvates thereof.

4. The method of claim 1, wherein the subject exhibits Postural Instability and Gait Difficulty (PIGD) subtype assessed by the MDS-UPDRS prior to administering to the subject a therapeutically effective amount of intepirdine or pharmaceutically acceptable salts, hydrates or solvates thereof.

5. The method of claim 1, wherein the neurodegenerative disease is selected from the group consisting of Alzheimer's disease, Creutzfeldt-Jakob disease, Dementia with Lewy bodies, frontotemporal dementia, Huntington's disease, levodopa-induced dyskinesia, Lewy body dementia, mixed Alzheimer's disease and cardiovascular disease, multiple sclerosis, orthostatic hypotension in Parkinson's disease, Parkinson's disease, Parkinson's disease dementia, progressive supranuclear palsy, vascular dementia, and any combination thereof.

6. The method of claim 1, wherein the subject is aged between about 50 and about 95 years of age.

7. The method of claim 1, wherein the subject is receiving stable therapy for a movement disorder, a balance disorder, a cognitive disorder, or any combination thereof.

8. The method of claim 7, wherein the subject is receiving a daily dose of about 1 mg to about 25 mg of donepezil.

9. The method of claim 1, wherein the therapeutically effective amount is between about 0.001 mg and 70 mg.

10. The method of claim 1, wherein the therapeutically effective amount is about 15 mg.

11. The method of claim 1, wherein the therapeutically effective amount is about 35 mg.

12. The method of claim 1, wherein the therapeutically effective amount is about 70 mg.

13. The method of claim 1, wherein administration of a therapeutically effective amount of intepirdine, or pharmaceutically acceptable salts, hydrates or solvates thereof, results in an improvement in quantitative measures of balance and gait.

14. The method of claim 1, wherein administration of a therapeutically effective amount of intepirdine, or pharmaceutically acceptable salts, hydrates or solvates thereof, results in an improvement in balance.

15. The method of claim 1, wherein administration of a therapeutically effective amount of intepirdine, or pharmaceutically acceptable salts, hydrates or solvates thereof, results in an improvement in movement and balance.

16. The method of claim 1 wherein administration of a therapeutically effective amount of intepirdine, or pharmaceutically acceptable salts, hydrates or solvates thereof, results in an improvement in balance confidence.

17. The method of claim 1, wherein administration of a therapeutically effective amount of intepirdine, or pharmaceutically acceptable salts, hydrates or solvates thereof, results in an improvement in gait.

18. The method of claim 1, wherein administration of a therapeutically effective amount of intepirdine, or pharmaceutically acceptable salts, hydrates or solvates thereof, results in an improvement in gait speed.

19. The method of claim 1, wherein administration of a therapeutically effective amount of intepirdine, or pharmaceutically acceptable salts, hydrates or solvates thereof, results in an improvement in gait variability.

20. The method of claim 1 wherein administration of a therapeutically effective amount of intepirdine, or pharmaceutically acceptable salts, hydrates or solvates thereof, results in a decrease in freezing of gait.

21. The method of claim 1, wherein administration of a therapeutically effective amount of intepirdine, or pharmaceutically acceptable salts, hydrates or solvates thereof, results in an improvement in the subjects' Motor Disorder Society Unified Parkinson's Disease Rating Scale (MDS-UPDRS), MDS-UPDRS Part III sub-score, MDS-UPDRS PIGD score, or any combination thereof.

22. The method of claim 1 wherein administration of a therapeutically effective amount of intepirdine, or pharmaceutically acceptable salts, hydrates or solvates thereof, results in an improvement in dynamic gait and balance.

23. The method of claim 1 wherein administration of a therapeutically effective amount of intepirdine, or pharmaceutically acceptable salts, hydrates or solvates thereof, results in an improvement in functional mobility.
